(19) United States
(12) Patent Application Publication
(10) Pub. No.: US 2016/0231341 A1
(43) Pub. Date: Aug. 11, 2016

(54) MULTIDRUG ANALYSIS IN URINE BY LIQUID CHROMATOGRAPHY-TANDEM MASS SPECTROMETRY

(71) Applicant: Castle Medical, LLC, Smyrna, GA (US)

(72) Inventors: Sreedharan Lakshmi Narayanan, Atlanta, GA (US); Thomas David, Atlanta, GA (US)

(21) Appl. No.: 14/614,846

(22) Filed: Feb. 5, 2015

(51) Int. Cl. G01N 33/94

(52) U.S. Cl. CPC G01N 33/9486; G01N 33/9466; G01N 2333/924

(57) ABSTRACT

A fast and reliable method for the determination of multiple drugs and their metabolites belonging to different chemical and toxicological class from a biological sample is provided. The method involves mixing of biological sample with internal standards which does not require sample extraction or derivatization prior to analysis. Further the samples were analyzed by scheduled multiple reaction monitoring using liquid chromatography tandem mass spectrometer (LC-MS/MS).

### TECHNICAL FIELD

The subject matter disclosed herein relates to a method of detection and/or quantification of multiple drugs and/or metabolites from a sample of body fluid by liquid chromatography-tandem mass spectrometry in a single run.

### BACKGROUND

Promoting mental health and preventing mental and/or substance abuse disorders are fundamental to SAMHSA's mission to reduce the impact of behavioral health conditions in America's communities.

Mental and substance abuse disorders can have a powerful effect on the health of individuals, their families, and their communities. In 2012, an estimated 9.6 million adults aged 18 and older in the United States had a serious mental illness, and 2.2 million youth aged 12 to 17 had a major depressive episode during the year 2011. In 2012, an estimated 23.1 million Americans aged 12 and older needed treatment for substance abuse. These disorders are among the top conditions that cause disability and carry a high burden of disease in the United States, resulting in significant costs to families, employers, and publicly funded health systems. By 2020, mental and substance abuse disorders will surpass all physical diseases as a major cause of disability worldwide.

In addition, drug and alcohol abuse can lead to other chronic diseases such as diabetes and heart disease. Addressing the impact of substance use alone is estimated to cost Americans more than $600 billion each year.

Preventing mental and/or substance abuse disorders and related problems in children, adolescents, and young adults are critical to Americans' behavioral and physical health. Behaviors and symptoms that signal the development of a behavioral disorder often manifest two to four years before a disorder is present. In addition, people with a mental health issue are more likely to use alcohol or drugs than those not affected by a mental illness. If communities and families can intervene early, behavioral health disorders might be prevented, or symptoms can be mitigated.

Data have shown that early intervention following the first episode of a serious mental illness can make an impact. Coordinated, specialized services offered during or shortly after the first episode of psychosis are effective for improving clinical and functional outcomes.

In addition, the Institute of Medicine and National Research Council's Preventing Mental, Emotional, and Behavioral Disorders Among Young People report—2009 notes that cost-benefit ratios for early treatment and prevention programs for addictions and mental illness programs range from 1:2 to 1:10. This means a $1 investment yields $2 to $10 savings in health costs, criminal and juvenile justice costs, educational costs, and lost productivity.

In different areas of human activities people are confronted with the illegal use of doping in order to enhance the output of the doped species. In sports this can be the improvement of the endurance of the athlete (like the swimmer, cyclist, triathlist, etc.), or (also) of the animal used in the sport (like the horse). Breeders of animals are sometimes known to illegally use doping to enhance the breeding process or the breeding product (like a faster growth of the animal). In order to combat such activities, authorities need fast and reliable equipment in order to detect such abuse. Generally the determination of the use of drugs in doping control is performed by analyzing the used drug or its metabolites in the body fluid of the treated species.

Confirmation of identity of forensically relevant compounds, such as drugs of abuse, is a necessary step in medico-legal event controls of people involved in crimes, workplace accidents and driving under the influence of drugs (DUID). Plasma is a useful medium in determining the short-term use of illicit drugs and its analysis is mandatory in the case of DUID in many countries. Urine has been the sample of choice for monitoring drug abuses in workplaces and is subjective to strict regulations.

The guidelines from the US Substance Abuse and Mental Health Services Administration (SAMHSA), effective October 2010, require LC/MS/MS methods for confirmation of initial drug tests.

Several methods are available in literature for drug-detection and quantification in body fluids.

WO 2007/134711 discloses a comprehensive multi-dimensional gas chromatography mass spectroscopic method for determining drug-metabolite in a body fluid by prior treatment of body fluid with alkyl haloformate.

CN 100381812 discloses liquid chromatography-tandem mass spectrometry (LC-MS/MS) used for drug detection from urine, which involves sample pre-treating and solid phase extraction. The method is reliable in the quantative detection of 19 drugs.

Journal article ‘Neurology (1972), 22(5), 540-50, discloses determination of multiple anticonvulsant drug levels in human serum by gas-liquid chromatography.

Journal article ‘Forensic Science International (2005), 150(2-3), 227-238’, discloses analysis of multiple illicit basic drugs in preserved oral fluid by solid-phase extraction and liquid chromatography-tandem mass spectrometry.

Journal article ‘Talanta (2009), 78(2), 377-387’ discloses use of ultra high-pressure liquid chromatography with a single quadrupole mass spectrometer for investigation of several cytochromes P 450 (CYP450) substrates and respiratory metabolites.

Journal article ‘Chromatographia (2012), 75(1-2), 55-63′ discloses determination of illicit drugs in urine and plasma by micro-SPE followed by HPLC-MS/MS.

Journal article ‘Archives of Pharmacal Research (2014), 37(6), 760-772’ discloses a method of screening multiple drugs of abuse and metabolites in urine using LC/MS/MS with polarity switching electrospray Ionization. This method involves simultaneous analysis of 35 drugs of abuse and relevant metabolites. The drugs and metabolites in urine were extracted by using mixed mode strong cation exchange polymeric solid phase extraction cartridges after enzymic hydrolysis and were then injected into the LC/MS/MS system.

Journal article reference ‘Journal of Analytical Toxicology (2007), 31(7), 359-368’ discloses determination of multiple drugs of abuse and relevant metabolites in urine by LC-MS-MS. The method is developed for the quantitative analysis of 30 drugs from classes such as opiates, barbiturates, amphetamines, cocaine, cannabinoids, phencyclidine, methadone, and benzodiazepines. This method uses solid-phase extraction (SPE) on an Oasis HLB column followed by liquid chromatography-tandem mass spectrometry.

Journal article reference ‘Rapid Communications in Mass Spectrometry (2014), 28(19), 2043-2053, discloses identification of multiple drugs of abuse and relative metabolites in urine samples using liquid chromatography/triple quadrupole mass spectrometry coupled with a library search with two multiple reaction monitoring (MRM) transitions per compound. The quantification and identification performance for 13 drugs of abuse and their metabolites were evaluated.

Journal article reference ‘Chromatographia (2001), 54(5/6), 345-349’ discloses method for simultaneous determination of multiple antiepileptic drugs in human serum.

Journal article reference ‘Journal of Mass Spectrometry (2008), 43(7), 980-992’ discloses multicomponent screening method for diuretics, masking agents, central nervous system (CNS) stimulants and opiates in human urine by UPLC-MS/MS.

Thus, the prior art methods of determining multiple drugs of abuse and their metabolites requires the special apparatus for extraction for sample preparation. In some prior art it is necessary to perform the derivatization of analyte, which subsequently will add to the cost and time for analysis. Though prior art methods discloses multiple drug analysis and their detection, the number of drugs and metabolites analyzed is limited to the class of drugs and number of drugs.

As a result, there is a need to provide a simple, cost effective, improved, highly efficient, and reliable method for quantitative analysis of a large number of drug or metabolites belonging to different chemical and toxicological classes in a biological sample.

The disclosed methods provide a rapid and cost effective method of drug-metabolite detection belonging to different chemical and toxicological classes form the body fluids such as urine. The disclosed methods meet SAMHSA guidelines to demonstrate linearity, limit of detection (LOD), accuracy and precision, as well as measurement of matrix effect, extraction recovery and overall process efficiency. Methods disclosed herein are suitable for all classes of SAMHSA-regulated drug.

### SUMMARY

In view of the problems of the related art discussed above, disclosed herein is an improved method of quantitative analysis of a drug or a metabolite in a biological sample. Moreover, an improved method of analysis is provided that is relatively salt free, which is important for mass spectrometry analysis.

The disclosed method facilitates the detection of 63 different drugs belonging to different chemical and toxicological classes in urine by liquid chromatography-tandem mass spectrometry method.

The disclosed method pertains to a sample preparation method that may be used for the quantitative analysis of a drug and/or metabolite in a biological sample. Moreover, also disclosed herein is a method for preparing a sample, without use of derivatization and/or extraction procedure, for quantitative analysis of a drug and/or metabolite in a biological sample.

The disclosed method can be a fast and reliable confirmatory method for the determination of multiple drugs of abuse belonging to different chemical and toxicological classes: opiates/opioids (28)-selected from 6-Monoacetylmorphine (6-MAM), Codeine, Dihydrocodeine, Hydrocodone, Hydromorphone, Morphine, Oxycodone, Oxymorphone, Buprenophrine, Carisoprodol, Desmethyl Tapentadol, Desmethyl Tramadol, 2-ethylidene-1,5-dimethyl-3,3-diphenylpyrrolidine (EDDP), Meperidine, Meprobamate, Methadone, Norbuprenophrine, Normeperidine, Tapentadol, Tramadol, Fentanyl, Norfentanyl, Norpropoxyphene, Propoxyphene, Dextromethophan, Dextrophan, Desomorphine, Nalaxone; benzodiazepines (12)-7-AminoClonazepam, Diazepam, Flunitrazepam, 4-HydroxyAlprazolam, Nordiazepam, Oxazepam, Temazepam, Chloradiazepoxide, OH-et-flunizepam, Lorazepam, Triazolam, Midazolam; barbiturates (2)-Butalbital, Phenobarbital; amphetamines (4)-Amphetamine, 3,4-methylenedioxyamphetamine (MDA), 3,4-methylenedioxy-methamphetamine (MDMA), Methamphetamine; tricyclic antidepressants (8)-Desipramine, Imipramine, Nortriptyline, Ritalinic Acid, Sertraline, Cyclobenzaprine, Amitriptyline, Methyl phenidate; illicit drugs (3) Tetrahydrocannabinolic acid (THCA), Benzoylecgonine, Phencyclidine (PCP); Z drugs (4)-Zolpidem, Zaleplon, Zopiclone, Zolpidem-COOH and Antiepileptics (2)-Pregabilan, Gabapentin.

### BRIEF DESCRIPTION OF THE DRAWINGS

FIG. 1 is an optimized multi-step gradient elution method for LC-MS/MS analysis. FIG. 2 is a scheduled MRM transitions in positive and negative mode. FIG. 3, panels labeled A, is a 7-points calibration curve for 6-Monoacetylmorphine (6-MAM) (left panel) and Buprenorphine (right panel) in positive mode. FIG. 3, panels labeled B, is a 7-points calibration curve for THCA (left panel) and Butalbital (right panel) in negative mode.

### DETAILED DESCRIPTION

Advantages of the present invention will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. The invention will be described in more detail hereinbelow by making reference to its particularly preferred embodiments.

Disclosed herein is highly selective and specific method for detection or determination of multiple drugs of abuse potential and their metabolite species belonging to different chemical and toxicological classes from a sample of body fluid comprising the steps: a) mixing the sample with internal standard, b) hydrolyzing the drug metabolite in the sample by β-Glucuronidase enzyme, c) centrifugation of the mixture of step (b) and diluting the clear supernatant liquid with deionized water and (d) analyzing said sample using liquid chromatography tandem mass spectrometer (LC-MS-MS) to determine the concentration of different drug metabolites; wherein, the method is devoid of derivatization and/or solid/liquid phase extraction of samples.

The term “accuracy” is art-recognized and describes the degree of conformity of a measure, i.e., the quantity, to a standard or a true value. For example, an increase in the accuracy of analyte quantification refers to an improvement in obtaining a measured value that is closer to the actual or true value. This improvement may be identified/described by reference to a percent increase in accuracy with respect to the accuracy obtainable using existing methods of measurement.

The term “analyte” as used herein, refers to any chemical or biological compound or substance that is subject to the analysis of the disclosed methods. Analytes of the disclosed methods include, but are not limited to, small organic compounds, amino acids, peptides, polypeptides, proteins, nucleic acids, polynucleotides, biomarkers, synthetic or natural polymers, or any combination or mixture thereof.

The term “analyte derivative,” as used herein, describes an analyte that is functionalized with another moiety in order to convert the analyte into a derivative thereof. It is the analyte derivative that is detected for use in determining the unknown quantity of an analyte in a sample, using a response factor calculation.

The term “analyzing” or “analysis” is used herein to describe the method by which the quantity of each of the individual analytes described herein is detected. Such analysis may be made using any technique that distinguishes between the analyte (or analyte derivative) and the analyte standard (or analyte derivative standard). In one embodiment of the disclosed methods, the analysis or act of analyzing includes liquid chromatography-tandem mass spectrometry (LC-MS-MS).

The term “chromatographic separation” is art-recognized, and describes the process in which a chemical mixture carried by a liquid or gas is separated into components as a result of differential distribution of the solutes as they flow around or over a stationary liquid or solid phase. For example, chromatographic separations suitable for use in the disclosed methods include, but are not limited to liquid chromatographic (including HPLC) methods such as normal-phase HPLC, RP-HPLC, HILIC, and size-exclusion chromatography (SEC), including gel permeation chromatography (GPC). Other suitable methods include additional HPLC methods and related liquid chromatographic techniques, including, e.g., ultra-performance liquid chromatography (HPLC), fast performance liquid chromatography (FPLC) and the like.

The term “internal standard,” as used herein, describes a collection of one or more functionalized chemical or biological compounds or substances, e.g., one or more analytes functionalized with another moiety in order to convert such compounds or substances into a derivative thereof. Internal standards of the disclosed methods are present in known concentrations and added to the sample to form a sample mixture. The addition of the internal standard allows for the detection of and comparison between the known concentrations of one or more known analytes, with the unknown concentrations of analytes in the original sample. As such, the internal standards of the disclosed methods provide a novel way to measure the absolute quantity of an analyte in sample using a response factor calculation.

The term “liquid chromatography” is art-recognized and includes chromatographic methods in which compounds are partitioned between a liquid mobile phase and a solid stationary phase. Liquid chromatographic methods are used for analysis or purification of compounds. The liquid mobile phase can have a constant composition throughout the procedure (an isocratic method), or the composition of the mobile phase can be changed during elution (e.g., a gradual change in mobile phase composition such as a gradient elution method).

The term “mass spectrometry” and “mass spectroscopy” are art-recognized and used herein, interchangeably to describe an instrumental method for identifying the chemical constitution of a substance by means of the separation of gaseous ions according to their differing mass and charge. A variety of mass spectrometry systems can be employed to analyze the analyte molecules of a sample subjected to the methods disclosed herein. For example, mass analyzers with high mass accuracy, high sensitivity and high resolution may be used and include, but are not limited to, atmospheric chemical ionization (APCI), chemical ionization (CI), electron impact (EI), fast atom bombardment (FAB), field desorption/field ionization (FD/FI), electrospray ionization (ESI), thermospray ionization (TSP), matrix-assisted laser desorption (MALDI), matrix-assisted laser desorption time-of-flight (MALDI-TOF) mass spectrometers, ESI-TOF mass spectrometers, and Fourier transform ion cyclotron mass analyzers (FT-ICR-MS). In addition, it should be understood that any combination of MS methods could be used in the methods described herein to analyze an analyte in a sample. In certain embodiments, the MS technique used for analysis of the analyte described herein is one that is applicable to most polar compounds, including amino acids, e.g., ESI.

The term “mobile phase” is art-recognized, and describes a liquid solvent system used to carry a compound of interest into contact with a solid phase (e.g., a solid phase in a solid phase extraction (SPE) cartridge or HPLC column) and to elute a compound of interest from the solid phase.

The term “precision” is art-recognized and describes the reproducibility of a result. It is measured by comparison of successive values obtained for a measurement to the prior values, where more precise measurements (or those with greater precision) will be demonstrated by successive measurements that are more consistently closer to the prior measurements.

The terms “quantitative” and “quantitatively” are art-recognized and used herein to describe measurements of quantity or amount. For example, the term “quantification” describes the act of measuring the quantity or amount of a particular object, e.g., an analyte. However, in the embodiments of the disclosed methods, the quantitative analysis is a measurement of an absolute amount, as opposed to relative amount, i.e., the total amount of analyte may be quantified absolutely in order to determine the actual amount of the analyte.

The term “sample” is used herein to describe a representative portion of a larger whole or group of components that are capable of being separated and detected by the methods disclosed herein. Exemplary samples include chemically or biologically derived substances, e.g., analytes of the disclosed methods. In particular embodiments, the components of the sample include, but are not limited to small organic compounds, amino acids, peptides, polypeptides, proteins, nucleic acids, polynucleotides, biomarkers, synthetic or natural polymers, or any combination or mixture thereof.

The term “sample mixture,” as used herein, describes the resultant product when a sample is mixed or combined with one or more analyte derivative standards, e.g., of a known concentration.

The term “Standard” as used herein, describes a reference material possessing one or more properties that are sufficiently well established that it can be used to prepare calibrators.

The term “Calibrator” as used herein, describes a solution, either prepared from the reference material or purchased, used to calibrate the assay. Where possible, calibrators should be prepared in a matrix similar to that of the specimens.

The term “Control” as used herein, describes a solution either prepared from the reference material (separately from the calibrators; that is, weighed or measured separately), purchased, or obtained from a pool of previously analyzed samples. Controls from any of these sources are used to determine the validity of the calibration; that is, the stability of a quantitative determination over time. Where possible, controls should be matrix-matched to specimens and calibrators, as indicated above.

The term “β-Glucuronidases” as used herein, describes a routinely used for the enzymatic hydrolysis of glucuronides from urine, plasma, and other fluids prior to analysis by enzyme immunoassay, mass spectrometry, gas chromatography, high performance liquid chromatography, or other means. Typically, between 1 and 20 units of glucuronidase is used per μl of plasma, urine, or bile for the enzymatic hydrolysis of glucuronides present in these samples. The exact amount needed will depend on the specific conditions used.

According to the methods disclosed herein the sample is a bodily fluid selected from the group consisting of oral fluids (saliva), sweat, urine, blood, serum, plasma, spinal fluid, and combination thereof.

According to the methods disclosed herein liquid chromatography tandem mass spectrometer (LC-MS-MS) comprises matrix-assisted laser desorption/ionization (MALDI) time-of-flight (TOF) MS analysis or electrospray ionization (ESI) MS.

In one another embodiment the methods disclosed herein involve a method for determination of drug/metabolite in a urine sample including:

(a) the sample were treated with enzyme and internal standard and mixture is allowed to react at room temperature

(b) calibrators were prepared in synthetic urine,

(c) detecting or determining the drug-metabolite by LC-MS-MS.

In some embodiments, the samples are independently selected from a saliva sample, a blood sample, a serum sample, a plasma sample, or a urine sample. There can be numerous advantages of the disclosed methods, such as
1. Rapid analysis process—8 minute/run
2. High number of drugs analyzed concurrently—63 drugs
3. Polarity switching for basic and acidic compounds
4. Sample volume required—100 μL
5. Results show high accuracy and precision for all the analytes
6. No extraction or derivatization steps required
7. Significantly reduced cost by using less consumables and reagents
8. Reduced man hours necessary for sample preparation
9. Reduced risk for errors in sample mix-ups and cross contamination

The advantages of this fast polarity switching, robust, sensitive, and rapid method yield a positive impact on a laboratory's goals in providing an accurate and lean process for the analysis of multiple drugs in a single run.

### EXAMPLES

### Multidrug Analysis from Urine

Standards, deuteriated internal standards (IS) were purchased from Cerilliant, Inc. and standard stock solution is prepared with all the analytes of interest at appropriate concentrations in methanol and stored in the freezer. Five levels of working standards are prepared form the stock solution and working standards in aliquots are stored as per manufactures recommendations, and are stable until manufactures listed expiration date.

Deuteriated internal standards (IS) are purchased from Cerilliant, Inc and are diluted to appropriate concentrations from which a stock IS solution is prepared. The stock contains 45 spiked with different concentrations. Aliquots of stock IS mix are kept at −4° C. Working IS prepared to acetate buffer and was stable for up to 30 days.

Negative control was purchased from Utak Laboratories Inc. Positive Threshold control (in house control prepared from Cerilliant standards with different lot numbers from the Calibrators). Pain Management control (PM 100) was purchased from Utak Laboratories and reconstituted as per manufacturer's instructions. Benzodiazepines (100 ng/ml) were obtained from Utak Laboratories. Bio-Rad Urine Toxicology Controls are employed for selected assays. A range of acceptable concentrations (±30%) was calculated from a series of assays and recorded.

All other chemicals and solvents were of the highest purity available from commercial sources and used without further purification.

### Method:

Urine samples were treated with P-Glucuronidase followed by minimum 90-minute incubation before centrifuging. Calibration standards and controls contain opiates/opioids (30); benzodiazepines (12); barbiturates (2) amphetamines (4); tricyclic antidepressants (8); illicit drugs (3); and Z drugs (4). Deuterated standards were used as the internal standards in the procedure. High performance liquid chromatography (Schimadzu LC-20) separation utilized gradient elution with a total run time of 8 minutes including a post run equilibration. Using positive and negative mode of ionization, an ABSciex 4500 triple-quadruple mass spectrometer was used to monitor the precursor and major product ions for each drug. Sequential MRM mode allowed for monitoring of multiple transitions based on an analyte specific retention time window. When a polarity switching experiment was utilized to obtain the maximum amount of information from a single injection, overall data quality was comparable to dedicated positive or negative experiments when intensity, signal-to-noise and reproducibility were compared. The number of data points measured across a chromatographic peak proved to be significantly high enough to achieve good resolution, precision and accuracy. Mobile phase comprised of water, methanol and acetonitrile with 0.1% Formic acid and 0.1% ammonium formate. Data analysis was performed on the Multiquant software version 3.0.

### Sample Preparation

The calibrators, controls and the sample prepared in the same method. All the samples and the working solutions (calibrators, controls, and internal standards) should be allowed to get into room temperature.
a) Qualitatively aliquot 100 μL of the calibrators, controls, and specimens into the vial and label the tubes.
b) If a dilution is necessary, record the dilution factor, volume of negative urine, and the volume of the specimen used to make the dilution on the work list.
c) Add 100 μL of the working internal standard (made of Acetate buffer pH 4.1) to the vial.
d) Add 25 VL of β-Glucuronidase enzyme to the vial
e) Call all the tubes and vortex
f) Incubate all tubes in 55° C. in the oven for 1.5 hrs.
g) Remove all the tubes from the oven to allow cooling to room temperature
h) Centrifuge the tubes at 14,000 RPM for 15 minutes
i) Transfer 100 μL to a clean vial and add 500 μL of DI H2O
j) Vortex and analyze by LC/MS/MS

### Instruments and Equipment

### LC Conditions:

The chromatographic separation and detection were performed using Kinetex 2.6u XB-C18 100A (50×3.0 mm) column (Phenomenox, USA). A mutli-step gradient elution method with an aqueous DI H2O containing 0.1% formic acid & ammonium formate (Mobile phase A) and 50% Acetonitrile & 50% Methanol containing 0.1% formic acid (Mobilephase B) at a flow rate of 0.50 mL/min to separate all the 63 compounds in positive and negative ionization mode. With a low sample injection volume of 10 μL and no sample preconcentraion, the presented method demonstrated excellent signal-to-noise (S/N) ratios due to the enhanced sensitivity of the Absicex 4500 Triple Quadrupole LC/MS/MS.

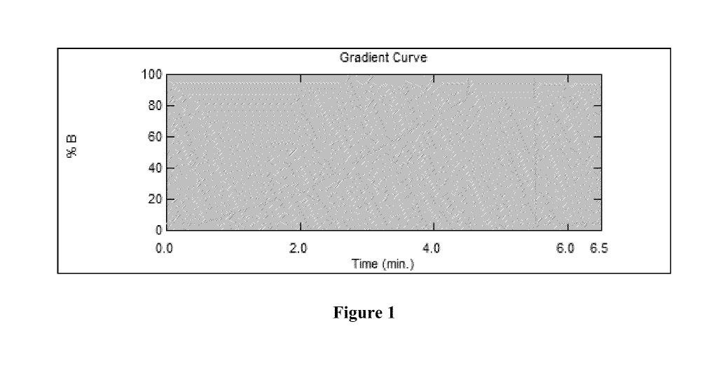

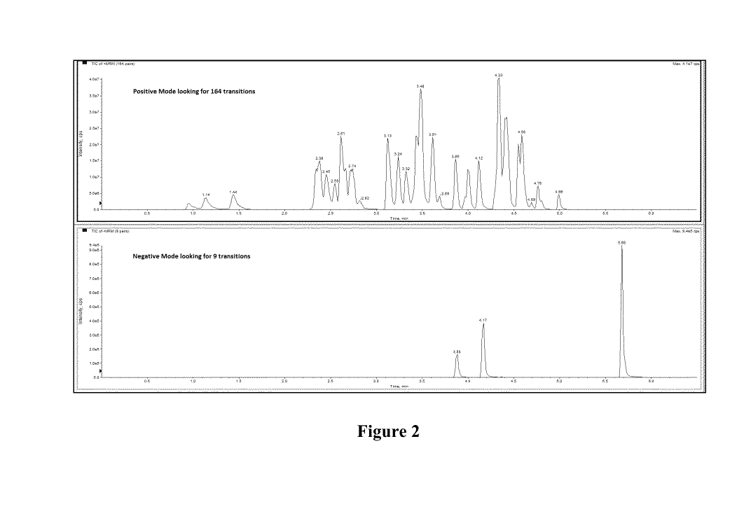

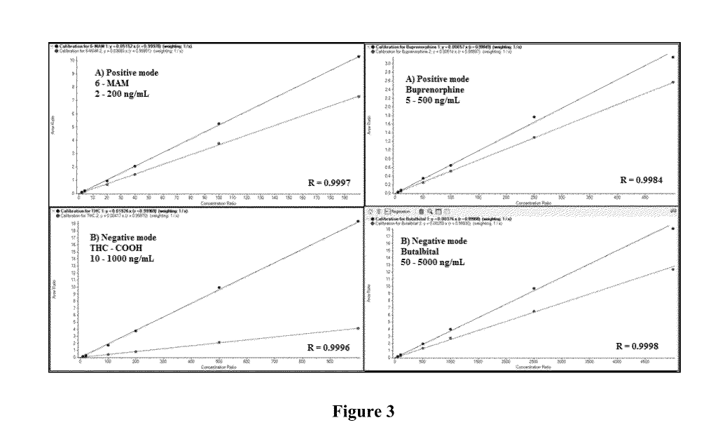

Column temperature set at 45° C. during whole run and the injection volume as 10 μL. The gradient elution method for chromatographic separation is provided in Table 1. The graphical view of optimized multi-step gradient elution method for LC/MS/MS is shown in FIG. 1.

TABLE 1
Gradient elution method for the separation

| Time | | |
|---|---|---|
| (mins) | Pump A | Pump B |
| 0.5 | 95% | 5% |
| 2.00 | 75% | 25% |
| 4.50 | 15% | 85% |
| 4.51 | 2% | 98% |
| 5.50 | 2% | 98% |
| 5.51 | 95% | 5% |
| 6.50 | 95% | 5% |

### Mass Spectrometer Conditions

ABsciex source provides a range of capabilities for testing samples. The precursor and product ions, along with optimized fragment and collision energy and other optimal voltages for each of the analytes are described in the method program. The ABSciex-Triple Quadrupole System consists of an ion source, enhanced desolvation technology followed by ion optics that transfer the ions to the first quadrupole (the Precursor Quad filter Q1), to the collision cell (Q2), and then to the third quadrupole (product Quad Filter Q3). The non-reactive inert gas nitrogen is used as collision gas. The precursor ion is selected using the first quadrupole and is sent to the collision cell for fragmentation. Fragment ions are derived from the precursor and therefore represent the structure of the precursor molecule. A specific precursor ion and specific product ions are thus selected and monitored. This type of analysis is termed Selected Reaction Monitoring (SRM). A triple quadrupole MS instrument running multiple SRMs for the same precursor ions is called Multiple Reaction Monitoring (MRM).

The Analyst software, a quantitation Wizard Program, is used to quantitate the analytes. A quantitation method is created with an algorithm (Intelliquan) which will generate quantitation tables. Integration of the peaks, regression and linearity of the calibration curve and accuracy of the standards and controls must be reviewed and verified.

The MS parameters for each analyte are obtained by infusing 10 ng/mL and optimized the parameters based on the sensitivity. The optimized parameters for the mass spectrometry were given in the following Table 2.

The specific parameters such as Declustering Potential (DP) decluster ions, Entrance Potential (EP), Collision Cell Entrance Potential (CE) and Collision Cell exit potential (CXP), parent ion, daughter ion and the retention time are mentioned in the Table 3 with 60 analytes in positive mode and 3 analytes in negative mode.

TABLE 2
Optimized parameter for the mass spectrometry.

| Parameters | Ranges |
|---|---|
| Curtain gas (CUR) | 35 |
| Collision Gas (CAD) | 7 |
| Ionspray Voltage (IS) | 2500 |
| Temperature (TEM) | 550 |
| Ion source Gas 1 (GS1) | 60 |
| Ion Source Gas 2 (GS 2) | 60 |

Determination: the sample were analyzed by liquid chromatography-tandem quadrupole mass spectrometer measured which enables 63 kinds of drugs belonging to different chemical and toxicological class, where barbiturates were analyzed in negative ion mode of analysis and all other compounds were analyzed using positive ion mode of analysis.

MS conditions and parameters for positive ion mode and negative ion mode can be used as are known in the art without affecting the overall concept of the methods disclosed herein.

TABLE 3
List of Internal Standards (IS) with Q1, Q3 and their
parameters for both positive and negative mode

| Internal Standard | Q1 | Q3 | RT | | | | |
|---|---|---|---|---|---|---|---|
| (IS) | (m/z) | (m/z) | (min) | DP | EP | CE | CXP |
| 6-MAM-D6 | 334.3 | 165.2 | 2.8 | 61.0 | 10.0 | 49.0 | 14.0 |
| Hydrocodone-D6 | 306.2 | 202.2 | 2.7 | 81.0 | 10.0 | 39.0 | 4.0 |
| Hydromorphone-D3 | 289.2 | 128.1 | 1.5 | 81.0 | 10.0 | 73.0 | 4.0 |
| Morphine-D3 | 289.2 | 152.1 | 1.0 | 66.0 | 10.0 | 77.0 | 4.0 |
| Oxycodone-D3 | 319.1 | 244.1 | 2.6 | 81.0 | 10.0 | 39.0 | 7.0 |
| Oxymorphone-D3 | 305.2 | 230.0 | 1.2 | 70.0 | 10.0 | 35.0 | 16.0 |
| Carisoprodol-D7 | 268.1 | 183.1 | 4.5 | 56.0 | 10.0 | 13.0 | 15.0 |
| Tapentadol-D3 | 225.2 | 121.0 | 3.4 | 51.0 | 10.0 | 27.0 | 4.0 |
| EDDP-D3 | 281.3 | 234.2 | 4.1 | 61.0 | 10.0 | 39.0 | 4.0 |
| Meperidine-D4 | 252.2 | 224.2 | 3.6 | 46.0 | 10.0 | 29.0 | 4.0 |
| Meprobamate-D7 | 226.2 | 165.3 | 3.8 | 41.0 | 10.0 | 11.0 | 4.0 |
| Methadone-D3 | 313.2 | 105.0 | 4.4 | 31.0 | 10.0 | 35.0 | 4.0 |
| Normeperidine-D4 | 238.2 | 164.2 | 3.6 | 36.0 | 10.0 | 23.0 | 4.0 |
| Tramadol-D3 | 267.2 | 58.0 | 3.4 | 60.0 | 10.0 | 75.0 | 8.0 |
| Fentanyl-D5 | 342.2 | 105.1 | 3.9 | 46.0 | 11.5 | 53.0 | 4.0 |
| Norfentanyl-D5 | 238.2 | 84.0 | 3.3 | 75.0 | 10.0 | 24.0 | 20.0 |
| Norpropoxyphene-D5 | 313.3 | 105.0 | 4.4 | 31.0 | 10.0 | 17.0 | 4.0 |
| Propoxyphene-D5 | 345.2 | 271.3 | 4.4 | 16.0 | 10.0 | 15.0 | 4.0 |
| 7-AminoClonazepam-D4 | 290.2 | 121.1 | 3.3 | 71.0 | 10.0 | 41.0 | 8.0 |
| Diazepam-D5 | 290.4 | 198.0 | 4.9 | 76.0 | 9.0 | 41.0 | 4.0 |
| 4-HydroxyAlprazolam-D5 | 330.2 | 302.1 | 4.5 | 86.0 | 10.0 | 27.0 | 4.0 |
| Nordiazepam-D5 | 276.1 | 165.1 | 4.7 | 71.0 | 10.0 | 39.0 | 10.0 |
| Oxazepam-D5 | 292.1 | 246.0 | 4.5 | 76.0 | 10.0 | 23.0 | 10.0 |
| OH-et-Flurazepam-D4 | 337.1 | 113.1 | 4.6 | 61.0 | 10.0 | 43.0 | 4.0 |
| Lorazepam-D4 | 325.2 | 279.0 | 4.6 | 76.0 | 6.5 | 25.0 | 4.0 |
| Zolpidem-D7 | 315.0 | 242.0 | 3.6 | 121.0 | 10.0 | 45.0 | 12.0 |
| Amphetamine-D6 | 142.2 | 93.1 | 2.6 | 41.0 | 10.5 | 21.0 | 4.0 |
| MDA-D5 | 185.2 | 168.0 | 2.7 | 41.0 | 10.0 | 29.0 | 4.0 |
| MDMA-D5 | 199.2 | 165.2 | 2.8 | 41.0 | 10.5 | 15.0 | 4.0 |
| Methamphetamine-D5 | 155.2 | 92.0 | 2.8 | 26.0 | 10.0 | 27.0 | 4.0 |
| Methylphenidate-D9 | 243.2 | 93.0 | 3.4 | 46.0 | 10.0 | 31.0 | 4.0 |
| Desipramine-D3 | 270.2 | 193.2 | 4.3 | 46.0 | 10.5 | 45.0 | 4.0 |
| Imipramine-D3 | 284.2 | 89.1 | 4.3 | 46.0 | 10.0 | 23.0 | 4.0 |
| Nortriptyline-D3 | 267.2 | 91.0 | 4.4 | 46.0 | 10.0 | 17.0 | 4.0 |
| Sertraline-D3 | 309.2 | 275.1 | 4.5 | 66.0 | 10.0 | 15.0 | 4.0 |
| Pregabalin-D6 | 166.0 | 148.1 | 2.4 | 40.0 | 10.0 | 13.0 | 6.0 |
| Gabapentin-D10 | 182.1 | 164.1 | 2.4 | 21.0 | 10.0 | 21.0 | 14.0 |
| Butalbital-D5 | 228.0 | 42.0 | 4.1 | −45.0 | −12.0 | −36.0 | −11.0 |
| Phenobarbital-D5 | 236.0 | 42.0 | 3.9 | −50.0 | −8.0 | −36.0 | −9.0 |
| THC-COOH D3 | 346.2 | 302.3 | 5.7 | −100.0 | −9.0 | −30.0 | −9.0 |
| Benzoylecgonine-D3 | 293.0 | 171.2 | 3.3 | 70.0 | 10.0 | 26.0 | 8.0 |
| PCP-D5 | 249.3 | 96.0 | 3.8 | 30.0 | 10.0 | 41.0 | 16.0 |

Q1—Quadrapole one,
Q3—Quadrapole three,
RT: Retention time,
DP—declustering potentials,
EP—entrance potentials,
CE—collision energies,
CXP—collision cell exit potentials,

TABLE 4
List of analytes with Q1, Q3 and their parameters for both positive and negative mode

| Drug-Metabolite | Drug -metabolite | Q1 | Q3 | RT | | | | |
|---|---|---|---|---|---|---|---|---|
| category | Name | (m/z) | (m/z) | (min) | DP | EP | CE | CXP |
| Opiates/opoids | 6MAM | 328.1 | 165.2 | 2.8 | 85.0 | 10.0 | 49.0 | 12.0 |
| | | 328.1 | 211.2 | 2.8 | 85.0 | 10.0 | 35.0 | 16.0 |
| | Codeine | 300.1 | 152.1 | 2.4 | 106.0 | 10.0 | 73.0 | 10.0 |
| | | 300.1 | 115.0 | 2.4 | 106.0 | 10.0 | 91.0 | 8.0 |
| | Dihydrocodeine | 302.3 | 128.2 | 2.4 | 66.0 | 10.0 | 81.0 | 4.0 |
| | | 302.3 | 199.1 | 2.4 | 66.0 | 10.0 | 41.0 | 4.0 |
| | Hydrocodone | 300.1 | 199.1 | 2.7 | 56.0 | 10.0 | 39.0 | 4.0 |
| | | 300.1 | 128.1 | 2.7 | 56.0 | 10.0 | 69.0 | 4.0 |
| | Hydromorphone | 286.2 | 185.0 | 1.5 | 56.0 | 10.0 | 39.0 | 4.0 |
| | | 286.2 | 128.0 | 1.5 | 56.0 | 10.0 | 73.0 | 4.0 |
| | Morphine | 286.2 | 152.0 | 1.0 | 56.0 | 10.0 | 73.0 | 4.0 |
| | | 286.2 | 165.0 | 1.0 | 56.0 | 10.0 | 47.0 | 4.0 |
| | Oxycodone | 316.1 | 241.0 | 2.6 | 50.0 | 10.0 | 43.0 | 15.0 |
| | | 316.1 | 256.0 | 2.6 | 50.0 | 10.0 | 38.0 | 15.0 |
| | Oxymorphone | 302.1 | 227.0 | 1.2 | 75.0 | 10.0 | 37.0 | 8.0 |
| | | 302.1 | 198.1 | 1.2 | 75.0 | 10.0 | 55.0 | 8.0 |
| | Buprenophrine | 468.3 | 396.1 | 4.1 | 55.0 | 10.0 | 53.0 | 14.0 |
| | | 468.3 | 414.2 | 4.1 | 55.0 | 10.0 | 43.0 | 6.0 |
| | Carisoprodol | 261.2 | 176.2 | 4.5 | 26.0 | 10.0 | 17.0 | 4.0 |
| | | 261.2 | 97.2 | 4.5 | 26.0 | 10.0 | 23.0 | 4.0 |
| | Desmethyl | 208.2 | 107.2 | 3.4 | 51.0 | 10.0 | 31.0 | 4.0 |
| | Tapentadol | 208.2 | 121.2 | 3.4 | 51.0 | 10.0 | 25.0 | 4.0 |
| | Desmethyl | 250.3 | 58.0 | 2.9 | 26.0 | 10.0 | 13.0 | 4.0 |
| | Tramadol | 250.3 | 91.0 | 2.9 | 26.0 | 10.0 | 55.0 | 4.0 |
| | EDDP | 278.2 | 234.2 | 4.1 | 100.0 | 10.0 | 67.0 | 4.0 |
| | | 278.2 | 249.2 | 4.1 | 100.0 | 10.0 | 43.0 | 4.0 |
| | Meperidine | 248.2 | 220.0 | 3.5 | 95.0 | 10.0 | 43.0 | 8.0 |
| | | 248.2 | 174.1 | 3.5 | 95.0 | 10.0 | 47.0 | 20.0 |
| | Meprobamate | 219.1 | 157.9 | 3.8 | 46.0 | 10.0 | 11.0 | 4.0 |
| | | 219.1 | 97.0 | 3.8 | 46.0 | 10.0 | 17.0 | 4.0 |
| | Methadone | 310.2 | 265.2 | 4.4 | 44.0 | 10.0 | 33.0 | 15.0 |
| | | 310.2 | 105.0 | 4.4 | 44.0 | 10.0 | 54.0 | 17.0 |
| | Norbuprenophrine | 414.1 | 211.0 | 3.8 | 95.0 | 10.0 | 50.0 | 8.0 |
| | | 414.1 | 187.0 | 3.8 | 95.0 | 10.0 | 45.0 | 8.0 |
| | Normeperidine | 234.1 | 160.2 | 3.6 | 36.0 | 10.0 | 21.0 | 4.0 |
| | | 234.1 | 188.1 | 3.6 | 36.0 | 10.0 | 17.0 | 4.0 |
| | Tapentadol | 222.1 | 107.2 | 3.4 | 116.0 | 10.0 | 39.0 | 18.0 |
| | | 222.1 | 121.2 | 3.4 | 116.0 | 10.0 | 27.0 | 12.0 |
| | Tramadol | 264.2 | 58.1 | 3.3 | 82.0 | 10.0 | 55.0 | 30.0 |
| | | 264.2 | 42.1 | 3.3 | 60.0 | 10.0 | 107.0 | 20.0 |
| | Fentanyl | 337.2 | 105.1 | 3.9 | 51.0 | 9.0 | 53.0 | 4.0 |
| | | 337.2 | 188.1 | 3.9 | 51.0 | 9.0 | 29.0 | 4.0 |
| | Norfentanyl | 233.2 | 84.1 | 3.3 | 41.0 | 10.0 | 23.0 | 4.0 |
| | | 233.2 | 150.0 | 3.3 | 41.0 | 10.0 | 23.0 | 4.0 |
| | Norpropoxyphene | 308.2 | 100.1 | 4.3 | 43.0 | 10.0 | 30.0 | 8.0 |
| | | 308.2 | 143.2 | 4.3 | 31.0 | 10.0 | 29.0 | 10.0 |
| | Propoxyphene | 340.2 | 266.3 | 4.3 | 57.0 | 10.0 | 19.0 | 23.0 |
| | | 340.2 | 91.1 | 4.3 | 16.0 | 10.0 | 67.0 | 14.0 |
| | Dextromethophan | 272.2 | 171.0 | 4.0 | 28.0 | 10.0 | 55.0 | 26.0 |
| | | 272.2 | 147.0 | 4.0 | 28.0 | 10.0 | 55.0 | 26.0 |
| | Dextrophan | 257.9 | 157.5 | 3.3 | 51.0 | 10.0 | 49.0 | 13.0 |
| | | 257.9 | 133.0 | 3.3 | 51.0 | 10.0 | 35.0 | 13.0 |
| | Desomorphine | 273.0 | 216.0 | 2.6 | 101.0 | 10.5 | 35.0 | 6.0 |
| | | 273.0 | 196.0 | 2.6 | 101.0 | 10.5 | 41.0 | 8.0 |
| | Nalaxone | 328.3 | 310.2 | 2.4 | 46.0 | 10.0 | 21.0 | 4.0 |
| | | 328.3 | 212.1 | 2.4 | 46.0 | 10.0 | 57.0 | 4.0 |
| Benzodiazepines | 7-AminoClonazepam | 286.0 | 121.2 | 3.3 | 68.0 | 10.0 | 36.0 | 16.0 |
| | | 286.0 | 222.1 | 3.3 | 68.0 | 10.0 | 34.0 | 14.0 |
| | Diazepam | 285.1 | 154.0 | 4.9 | 101.0 | 10.0 | 39.0 | 26.0 |
| | | 285.1 | 193.1 | 4.9 | 101.0 | 10.0 | 45.0 | 10.0 |
| | Flunitrazepam | 314.1 | 268.1 | 4.6 | 91.0 | 10.0 | 50.0 | 22.0 |
| | | 314.1 | 239.1 | 4.6 | 91.0 | 10.0 | 62.0 | 14.0 |
| | 4-HydroxyAlprazolam | 325.1 | 297.2 | 4.5 | 71.0 | 10.0 | 27.0 | 4.0 |
| | | 325.1 | 216.1 | 4.5 | 71.0 | 10.0 | 53.0 | 4.0 |
| | Nordiazepam | 271.0 | 140.0 | 4.7 | 71.0 | 10.0 | 37.0 | 10.0 |
| | | 271.0 | 165.1 | 4.7 | 71.0 | 10.0 | 37.0 | 14.0 |
| | Oxazepam | 287.0 | 241.1 | 4.5 | 76.0 | 10.0 | 33.0 | 11.0 |
| | | 287.0 | 269.1 | 4.5 | 76.0 | 10.0 | 23.0 | 10.0 |
| | Temazepam | 301.1 | 255.1 | 4.7 | 70.0 | 10.0 | 31.0 | 20.0 |
| | | 301.1 | 283.0 | 4.7 | 70.0 | 10.0 | 21.0 | 14.0 |
| | Chloradiazepoxide | 300.0 | 283.1 | 4.1 | 70.0 | 5.0 | 25.0 | 4.0 |
| | | 300.0 | 227.2 | 4.1 | 70.0 | 5.0 | 33.0 | 3.0 |
| | OH-et Flurazepam | 333.1 | 109.1 | 4.6 | 61.0 | 10.0 | 43.0 | 4.0 |
| | | 333.1 | 211.3 | 4.6 | 61.0 | 10.0 | 43.0 | 4.0 |
| | Lorazepam | 321.0 | 275.0 | 4.6 | 75.0 | 10.0 | 31.0 | 14.0 |
| | | 321.0 | 229.0 | 4.6 | 75.0 | 10.0 | 43.0 | 16.6 |
| | Triazolam | 344.9 | 317.0 | 4.6 | 111.0 | 10.0 | 37.0 | 14.0 |
| | | 344.9 | 310.0 | 4.6 | 111.0 | 10.0 | 35.0 | 14.0 |
| | Midazolam | 326.1 | 291.1 | 4.1 | 101.0 | 10.0 | 37.0 | 22.0 |
| | | 326.1 | 249.1 | 4.1 | 101.0 | 10.0 | 47.0 | 18.0 |
| Z drugs | Zolpidem | 308.0 | 235.1 | 3.6 | 85.0 | 10.0 | 65.0 | 45.0 |
| | | 308.0 | 219.0 | 3.6 | 51.0 | 10.0 | 77.0 | 6.0 |
| | Zaleplon | 306.1 | 264.0 | 4.3 | 66.0 | 10.0 | 21.0 | 4.0 |
| | | 306.1 | 236.0 | 4.3 | 66.0 | 10.0 | 45.0 | 4.0 |
| | Zopiclone | 389.2 | 244.9 | 3.3 | 15.0 | 10.0 | 27.0 | 10.0 |
| | | 389.2 | 217.0 | 3.3 | 15.0 | 10.0 | 45.0 | 8.0 |
| | Zolpidem-COOH | 337.8 | 293.1 | 3.1 | 126.0 | 10.0 | 37.0 | 12.0 |
| | | 337.8 | 265.1 | 3.1 | 126.0 | 10.0 | 47.0 | 13.0 |
| Amphetamines | Amphetamine | 136.1 | 91.0 | 2.6 | 64.0 | 10.0 | 33.0 | 12.0 |
| | | 136.1 | 119.0 | 2.6 | 60.0 | 10.0 | 17.0 | 8.0 |
| | MDA | 180.1 | 163.0 | 2.7 | 16.0 | 10.0 | 29.0 | 4.0 |
| | | 180.1 | 133.0 | 2.7 | 16.0 | 10.0 | 23.0 | 4.0 |
| | MDMA | 194.1 | 163.2 | 2.9 | 65.0 | 10.0 | 31.0 | 14.0 |
| | | 194.1 | 105.2 | 2.9 | 65.0 | 10.0 | 40.0 | 18.0 |
| | Methamphetamine | 150.1 | 91.2 | 2.8 | 53.0 | 10.0 | 42.0 | 38.0 |
| | | 150.1 | 119.2 | 2.8 | 50.0 | 10.0 | 22.0 | 19.0 |
| Tricyclic | Methyl phenidate | 234.3 | 84.1 | 3.4 | 60.0 | 10.0 | 38.0 | 20.0 |
| antidepressants | | 234.3 | 56.1 | 3.4 | 46.0 | 10.0 | 63.0 | 14.0 |
| | Desipramine | 267.3 | 72.1 | 4.3 | 46.0 | 10.0 | 33.0 | 20.0 |
| | | 267.3 | 193.1 | 4.3 | 46.0 | 10.0 | 45.0 | 4.0 |
| | Imipramine | 281.3 | 86.1 | 4.3 | 50.0 | 10.0 | 30.0 | 15.0 |
| | | 281.3 | 58.1 | 4.3 | 50.0 | 10.0 | 55.0 | 15.0 |
| | Nortriptyline | 264.2 | 117.2 | 4.4 | 56.0 | 10.0 | 25.0 | 4.0 |
| | | 264.2 | 233.2 | 4.4 | 56.0 | 10.0 | 17.0 | 4.0 |
| | Ritalinic Acid | 220.2 | 84.1 | 3.2 | 52.0 | 10.0 | 34.0 | 15.0 |
| | | 220.2 | 56.1 | 3.2 | 52.0 | 10.0 | 60.0 | 14.0 |
| | Sertraline | 306.1 | 159.1 | 4.5 | 30.0 | 11.0 | 33.0 | 14.0 |
| | | 306.1 | 275.1 | 4.5 | 30.0 | 11.0 | 17.0 | 17.0 |
| | Cyclobenzaprine | 276.2 | 215.0 | 4.3 | 90.0 | 10.0 | 50.0 | 8.0 |
| | | 276.2 | 216.2 | 4.3 | 90.0 | 10.0 | 35.0 | 8.0 |
| | Amitriptyline | 278.4 | 91.0 | 4.4 | 56.0 | 10.0 | 33.0 | 8.0 |
| | | 278.4 | 117.1 | 4.4 | 56.0 | 10.0 | 31.0 | 8.0 |
| Antiepileptics | Pregabalin | 160.1 | 142.0 | 2.4 | 40.0 | 10.0 | 23.0 | 4.0 |
| | | 160.1 | 97.0 | 2.4 | 40.0 | 10.0 | 23.0 | 4.0 |
| | Gabapentin | 172.1 | 95.1 | 2.4 | 64.0 | 10.0 | 30.0 | 15.0 |
| | | 172.1 | 137.2 | 2.4 | 64.0 | 10.0 | 21.0 | 13.0 |
| Barbiturates | Butalbital | 223.1 | 42.0 | 4.1 | −55.0 | −12.0 | −40.0 | −11.0 |
| | | 223.1 | 180.0 | 4.1 | −55.0 | −12.0 | −16.0 | −5.0 |
| | Phenobarbital | 231.1 | 42.0 | 3.9 | −55.0 | −8.0 | −42.0 | −5.0 |
| | | 231.1 | 188.0 | 3.9 | −55.0 | −8.0 | −14.0 | −9.0 |
| Illicit drugs | THCA | 343.1 | 299.1 | 5.7 | −115.0 | −9.0 | −30.0 | −11.0 |
| | | 343.1 | 245.1 | 5.7 | −115.0 | −9.0 | −40.0 | −13.0 |
| | Benzoylecgonine | 290.1 | 168.1 | 3.3 | 61.0 | 10.0 | 38.0 | 30.0 |
| | | 290.1 | 105.0 | 3.3 | 61.0 | 10.0 | 45.0 | 16.0 |
| | PCP | 244.2 | 91.0 | 3.8 | 21.0 | 10.0 | 41.0 | 4.0 |
| | | 244.2 | 86.0 | 3.8 | 21.0 | 10.0 | 41.0 | 4.0 |

Q1—Quadrapole one,
Q3—Quadrapole three,
RT: Retention time,
DP—declustering potentials,
EP—entrance potentials,
CE—collision energies,
CXP—collision cell exit potentials,

The limit of quantification varies from 2 to 100 ng/mL depends on the analytes. The list of analytes shown in the Table 5 based on the classification.

TABLE 5
Analytes based on classification

| Drug Name | LOQ |
|---|---|
| 6MAM | 2 |
| Codeine | 50 |
| Dihydrocodeine | 50 |
| Hydrocodone | 50 |
| Hydromorphone | 50 |
| Morphine | 50 |
| Oxycodone | 50 |
| Oxymorphone | 50 |
| Buprenophrine | 5 |
| Carisoprodol | 50 |
| Desmethyl Tapentadol | 50 |
| DesmethylTramadol | 50 |
| EDDP | 50 |
| Meperidine | 50 |
| Meprobamate | 50 |
| Methadone | 50 |
| Norbuprenophrine | 10 |
| Normeperidine | 50 |
| Tapentadol | 50 |
| Tramadol | 50 |
| Fentanyl | 2 |
| Norfentanyl | 2 |
| Norpropoxyphene | 50 |
| Propoxyphene | 50 |
| Dextromethophan | 50 |
| Dextrophan | 50 |
| Desomorphine | 50 |
| Nalaxone | 10 |
| 7-AminoClonazepam | 10 |
| Diazepam | 10 |
| Flunitrazepam | 10 |
| HydroxyAlprazolam | 10 |
| Nordiazepam | 10 |
| Oxazepam | 10 |
| Temazepam | 10 |
| Chloradiazepoxide | 10 |
| OH-et-Flurazepam | 10 |
| Lorazepam | 10 |
| Triazolam | 10 |
| Midazolam | 10 |
| Z-DRUGS | |
| Zolpidem | 10 |
| Zaleplon | 10 |
| Zopiclone | 10 |
| Zolpidem-COOH | 10 |
| TRICYCLIC | |
| ANTIDEPRESSANTS | |
| Desipramine | 20 |
| Imipramine | 20 |
| Methyl Phenidate | 50 |
| Nortriptyline | 20 |
| Ritalinic Acid | 100 |
| Sertraline | 20 |
| Cyclobenzaprine | 20 |
| Amitriptyline | 20 |
| ANTIEPILEPTICS | |
| Pregabilan | 50 |
| Gabapentin | 100 |
| AMPHETAMINES | |
| Amphetamine | 50 |
| MDA | 50 |
| MDMA | 50 |
| Methamphetamine | 50 |
| BARBITURATES | |
| Butalbital | 50 |
| Phenobarbital | 50 |
| ILLICITS | |
| THCA | 10 |
| Benzoylecgonine | 50 |
| PCP | 4 |

### Results:

All drugs were analyzed simultaneously using the same procedure steps. Target concentrations varied between 2 ng/ml to 20,000 ng/ml among the analytes depending on their therapeutic and toxic levels. Approximately 173 transitions were monitored per run by sequential MRM. Assays with low level cutoffs (such as 2 ng/mL for certain opioids such as fentanyl and 20 ng/ml for benzodiazepines) were reproducible and met acceptable chromatographic criteria. An accuracy of 95-105% and a CV of 2.0-10.2 were achieved for each calibration point in a method validation study. Further statistical data indicated an accuracy of 96-108% for quality control samples. The limit of detection and the upper linearity limit results have been quantified and established for each component.

### MRM Parameter

Each chemical compound supposes two female ions-sub-ion pair, is divided into 3 MRM channel collections.

The methods disclosed herein can achieve the lowest detection limit 2-200 ng/mL. Within 2-100 ng/mL concentration range, 63 kinds of drugs good linear correlation coefficient of 0.9771-0.9995. In urine, the recovery of most drugs between 70-130% and the recovery of a few drugs between 60-70%, RSD is less than 15.0%, to meet the needs of daily quantitative analysis.

SAMHSA guidelines require the use of one quantifier and at least one quantifier for both target compound and internal standard (Table 3) The detection methods disclosed herein to establish a liquid chromatography-tandem mass spectrometry detection in urine 63 kinds of common drugs. Can be used for Entry-Exit Inspection and Quarantine, Centers for Disease Control, the public security departments rapid detection and confirmation of positive results.

### LC-MS Set Up:

ABSciex-Triple Quadrupole System 4500 consists of an ion source, enhanced desolvation technology with Electron Spray Ionization in positive mode.

For all MS-MS experiments, mass calibration and resolution adjustments (at 0.7 amu full width at half height) on both the resolving quadrupoles were automatically optimized using a poly(propylene)glycol 1 3 1024 mol/L solution introduced via the built-in infusion pump. In the ABSciex API3200 Triple Quad adjustable voltage, Declustering Potential (DP) declusters ions, Entrance Potential (EP) focuses ions, Collision Cell Entrance Potential (CEP) focuses ions into Q2, Collision Energy fragments ions and Collision Cell exit potential (CXP) assists ions going into Q3. All these voltages are optimized for each analyte in the assay by compound optimization and the values are incorporated into the acquisition method.

TABLE 6
Chromatography parameters

| Parameters | Ranges | |
|---|---|---|
| Chromatography mode | Reverse phase | |
| Isocratic/gradient method | Gradient | |
| Aqueous phase | DI H2O with 0.1% Formic Acid | |
| | and 0.1% Ammonium Formate | |
| Organic phase | 50.0% Acetonitrile and 50.0% | |
| | Methanol with 0.1% Formic Acid | |
| Needle Wash | 40.0% Isopropanol + 40.0% | |
| | Acetonitrile + 20.0% Acetone | |
| Flow rate | 0.5 | mL/min |
| Run time | 6.5 | minutes |
| Sample injection volume | 10 | μL |
| Column temperature | 45° | C. |

TABLE 7
Ion source parameters

| Parameters | Ranges | |
|---|---|---|
| Interface | Electrospray ionization | |
| Ionization mode | Positive and negative | |
| Source/Gas temperature | 550° | C. |
| Ion source gas 1 | 60 | PSI |
| Ion source gas 2 | 60 | PSI |
| Curtain Gas | 35 | PSI |
| Collision gas | 7 | PSI |

| | |
|---|---|
| Ion Spray voltages | 2500 |

### Method Validation

Method was validated for linearity, accuracy, precision, recovery, LOD and LOQ.

Calibration curves for drug/metabolite were made by serial dilution from a stock solution and were created in duplicates. Peak heights vs nominal concentrations were used to construe calibration curves. Curves were evaluated using least squares fitting and by linear regression analysis.

The calibration for concentration from 2 ng/ml to 10000 ng/ml and its linearity is shown in Table 8.

TABLE 8
Linearity

| | | | | | | | | |
|---|---|---|---|---|---|---|---|---|
| No. | Drug Name | LOQ | Cal 1 | Cal 2 | Cal 3 | Cal 4 | Cal 5 | ULOL |

OPIATES

| | | | | | | | | |
|---|---|---|---|---|---|---|---|---|
| 1 | 6MAM | 2 | 4 | 20 | 40 | 100 | 200 | 200 |
| 2 | Codeine | 50 | 100 | 500 | 1000 | 2500 | 5000 | 5000 |
| 3 | Dihydrocodeine | 50 | 100 | 500 | 1000 | 2500 | 5000 | 5000 |
| 4 | Hydrocodone | 50 | 100 | 500 | 1000 | 2500 | 5000 | 10000 |
| 5 | Hydromorphone | 50 | 100 | 500 | 1000 | 2500 | 5000 | 10000 |
| 6 | Morphine | 50 | 100 | 500 | 1000 | 2500 | 5000 | 10000 |
| 7 | Oxycodone | 50 | 100 | 500 | 1000 | 2500 | 5000 | 10000 |
| 8 | Oxymorphone | 50 | 100 | 500 | 1000 | 2500 | 5000 | 10000 |
| 9 | Buprenophrine | 5 | 10 | 50 | 100 | 250 | 500 | 2000 |
| 10 | Carisoprodol | 50 | 100 | 500 | 1000 | 2500 | 5000 | 10000 |
| 11 | Desmethyl Tapentadol | 50 | 100 | 500 | 1000 | 2500 | 5000 | 10000 |
| 12 | DesmethylTramadol | 50 | 100 | 500 | 1000 | 2500 | 5000 | 10000 |
| 13 | EDDP | 50 | 100 | 500 | 1000 | 2500 | 5000 | 10000 |
| 14 | Meperidine | 50 | 100 | 500 | 1000 | 2500 | 5000 | 7500 |
| 15 | Meprobamate | 50 | 100 | 500 | 1000 | 2500 | 5000 | 10000 |
| 16 | Methadone | 50 | 100 | 500 | 1000 | 2500 | 5000 | 10000 |
| 17 | Norbuprenophrine | 10 | 20 | 100 | 200 | 500 | 1000 | 2000 |
| 18 | Normeperidine | 50 | 100 | 500 | 1000 | 2500 | 5000 | 10000 |
| 19 | Tapentadol | 50 | 100 | 500 | 1000 | 2500 | 5000 | 10000 |
| 20 | Tramadol | 50 | 100 | 500 | 1000 | 2500 | 5000 | 10000 |
| 21 | Fentanyl | 2 | 4 | 20 | 40 | 100 | 200 | 800 |
| 22 | Norfentanyl | 2 | 4 | 20 | 40 | 100 | 200 | 800 |
| 23 | Norpropoxyphene | 50 | 100 | 500 | 1000 | 2500 | 5000 | 10000 |
| 24 | Propoxyphene | 50 | 100 | 500 | 1000 | 2500 | 5000 | 10000 |
| 25 | Dextromethophan | 50 | 100 | 500 | 1000 | 2500 | 5000 | 5000 |
| 26 | Dextrophan | 50 | 100 | 500 | 1000 | 2500 | 5000 | 5000 |
| 27 | Desomorphine | 50 | 100 | 500 | 1000 | 2500 | 5000 | 5000 |
| 28 | Nalaxone | 10 | 20 | 100 | 200 | 500 | 1000 | 5000 |

BENZODIAZEPINES

| | | | | | | | | |
|---|---|---|---|---|---|---|---|---|
| 29 | 7-AminoClonazepam | 10 | 20 | 100 | 200 | 500 | 1000 | 5000 |
| 30 | Diazepam | 10 | 20 | 100 | 200 | 500 | 1000 | 5000 |
| 31 | Flunitrazepam | 10 | 20 | 100 | 200 | 500 | 1000 | 5000 |
| 32 | HydroxyAlprazolam | 10 | 20 | 100 | 200 | 500 | 1000 | 5000 |
| 33 | Nordiazepam | 10 | 20 | 100 | 200 | 500 | 1000 | 5000 |
| 34 | Oxazepam | 10 | 20 | 100 | 200 | 500 | 1000 | 5000 |
| 35 | Temazepam | 10 | 20 | 100 | 200 | 500 | 1000 | 5000 |
| 36 | Chloradiazepoxide | 10 | 20 | 100 | 200 | 500 | 1000 | 5000 |
| 37 | OH-et-flunizepam | 10 | 20 | 100 | 200 | 500 | 1000 | 5000 |
| 38 | Lorazepam | 10 | 20 | 100 | 200 | 500 | 1000 | 5000 |
| 39 | Triazolam | 10 | 20 | 100 | 200 | 500 | 1000 | 5000 |
| 40 | Midazolam | 10 | 20 | 100 | 200 | 500 | 1000 | 5000 |

Z-DRUGS

| | | | | | | | | |
|---|---|---|---|---|---|---|---|---|
| 41 | Zolpidem | 10 | 20 | 100 | 200 | 500 | 1000 | 5000 |
| 42 | Zaleplon | 10 | 20 | 100 | 200 | 500 | 1000 | 5000 |
| 43 | Zopiclone | 10 | 20 | 100 | 200 | 500 | 1000 | 5000 |
| 44 | Zolpidem-COOH | 10 | 20 | 100 | 200 | 500 | 1000 | 5000 |

TRICYCLIC ANTIDEPRESSANTS

| | | | | | | | | |
|---|---|---|---|---|---|---|---|---|
| 45 | Desipramine | 20 | 40 | 200 | 400 | 1000 | 2000 | 5000 |
| 46 | Imipramine | 20 | 40 | 200 | 400 | 1000 | 2000 | 5000 |
| 47 | MethylPhenydate | 50 | 100 | 500 | 1000 | 2500 | 5000 | 5000 |
| 48 | Nortriptyline | 20 | 40 | 200 | 400 | 1000 | 2000 | 5000 |
| 49 | Ritalinic Acid | 100 | 200 | 1000 | 2000 | 5000 | 10000 | 20000 |
| 50 | Sertraline | 20 | 40 | 200 | 400 | 1000 | 2000 | 5000 |
| 51 | Cyclobenzaprine | 20 | 40 | 200 | 400 | 1000 | 2000 | 5000 |
| 52 | Amitriptyline | 20 | 40 | 200 | 400 | 1000 | 2000 | 5000 |

ANTIEPILEPTICS

| | | | | | | | | |
|---|---|---|---|---|---|---|---|---|
| 53 | Pregabilan | 50 | 100 | 500 | 1000 | 2500 | 5000 | 10000 |
| 54 | Gabapentin | 100 | 200 | 1000 | 2000 | 5000 | 10000 | 10000 |

AMPHETAMINES

| | | | | | | | | |
|---|---|---|---|---|---|---|---|---|
| 55 | Amphetamine | 50 | 100 | 500 | 1000 | 2500 | 5000 | 10000 |
| 56 | MDA | 50 | 100 | 500 | 1000 | 2500 | 5000 | 10000 |
| 57 | MDMA | 50 | 100 | 500 | 1000 | 2500 | 5000 | 10000 |
| 58 | Methamphetamine | 50 | 100 | 500 | 1000 | 2500 | 5000 | 10000 |

BARBITURATES

| | | | | | | | | |
|---|---|---|---|---|---|---|---|---|
| 59 | Butalbital | 50 | 100 | 500 | 1000 | 2500 | 5000 | 10000 |
| 60 | Phenobarbital | 50 | 100 | 500 | 1000 | 2500 | 5000 | 10000 |

ILLICITS

| | | | | | | | | |
|---|---|---|---|---|---|---|---|---|
| 61 | THCA | 10 | 20 | 100 | 200 | 500 | 1000 | 1000 |
| 62 | Benzoylecgonine | 50 | 100 | 500 | 1000 | 2500 | 5000 | 10000 |
| 63 | PCP | 4 | 8 | 40 | 80 | 200 | 400 | 2000 |

### Precision

TABLE 9
Intraday accuracy and precision studied 10 runs

| | Actual | Mean | CV | |
|---|---|---|---|---|
| Component Name | (ng/mL) | n = 10 | (%) | Accuracy |
| 6-MAM | 2 | 1.85 | 10.85 | 92.28 |
| | 4 | 3.65 | 9.01 | 91.37 |
| | 20 | 18.21 | 11.23 | 91.07 |
| | 40 | 40.26 | 7.96 | 100.66 |
| | 100 | 91.66 | 8.94 | 91.66 |
| | 200 | 210.37 | 10.27 | 105.18 |
| OH-et-Flurazepam | 10 | 9 | 6.57 | 90 |
| | 20 | 19 | 9.65 | 95.01 |
| | 100 | 93.86 | 3.34 | 93.86 |
| | 200 | 192.06 | 4.03 | 96.03 |
| | 500 | 450.87 | 3.32 | 90.17 |
| | 1000 | 1065.2 | 5.14 | 106.52 |
| 4-Hydroxyalprazolam | 10 | 10.74 | 8.03 | 107.43 |
| | 20 | 20.64 | 6.22 | 103.21 |
| | 100 | 98.42 | 7.45 | 98.42 |
| | 200 | 193.36 | 7.21 | 96.68 |
| | 500 | 462.43 | 4.93 | 92.49 |
| | 1000 | 1044.4 | 8.52 | 104.44 |
| 7-Aminoclonazepam | 10 | 10.31 | 7.85 | 103.13 |
| | 20 | 19.77 | 8.26 | 98.87 |
| | 100 | 94.07 | 4.87 | 94.07 |
| | 200 | 203.54 | 8.48 | 101.77 |
| | 500 | 461.98 | 6.15 | 92.4 |
| | 1000 | 1040.33 | 6.09 | 104.03 |
| Amitriptyline | 20 | 21.82 | 7.21 | 109.08 |
| | 40 | 42.95 | 6.67 | 107.38 |
| | 200 | 193.08 | 4.32 | 96.54 |
| | 400 | 417.8 | 5.29 | 104.45 |
| | 1000 | 972.07 | 4.95 | 97.21 |
| | 2000 | 2012.28 | 2.28 | 100.61 |
| Amphetamine | 50 | 57.62 | 5.52 | 115.24 |
| | 100 | 121.11 | 7.26 | 121.11 |
| | 500 | 633.3 | 6.15 | 126.66 |
| | 1000 | 1226.36 | 3.48 | 122.64 |
| | 2500 | 2419.79 | 5.37 | 96.79 |
| | 5000 | 4599.11 | 6.75 | 91.98 |
| Benzoylecgonine | 50 | 59.19 | 5.61 | 118.37 |
| | 100 | 118.85 | 4.33 | 118.85 |
| | 500 | 576.98 | 5.38 | 115.4 |
| | 1000 | 1128.86 | 4.12 | 112.89 |
| | 2500 | 2510.59 | 4.23 | 100.42 |
| | 5000 | 4755.53 | 3.24 | 95.11 |
| Buprenorphine | 5 | 4.45 | 9.45 | 88.97 |
| | 10 | 9.85 | 12.31 | 98.45 |
| | 50 | 46.85 | 4.78 | 93.7 |
| | 100 | 94.57 | 6.19 | 94.57 |
| | 250 | 236.92 | 6.83 | 94.77 |
| | 500 | 522.37 | 6.19 | 104.47 |
| Carisoprodol | 50 | 58.55 | 7.65 | 117.1 |
| | 100 | 129.8 | 5.96 | 129.8 |
| | 500 | 608.97 | 9.30 | 121.79 |
| | 1000 | 1172.67 | 7.12 | 117.27 |
| | 2500 | 2524.38 | 7.13 | 100.98 |
| | 5000 | 4648.64 | 9.13 | 92.97 |
| Chlordiazepoxide | 10 | 8.01 | 9.73 | 80.13 |
| | 20 | 15.91 | 5.14 | 79.55 |
| | 100 | 77.2 | 6.72 | 77.2 |
| | 200 | 166.47 | 8.55 | 83.23 |
| | 500 | 448.7 | 6.63 | 89.74 |
| | 1000 | 1113.71 | 5.40 | 111.37 |
| Codeine | 50 | 47.91 | 11.12 | 95.82 |
| | 100 | 96.41 | 8.78 | 96.41 |
| | 500 | 479.82 | 9.72 | 95.96 |
| | 1000 | 936.27 | 7.70 | 93.63 |
| | 2500 | 2313.89 | 6.08 | 92.56 |
| | 5000 | 5275.7 | 8.47 | 105.51 |
| Cyclobenzaprine | 20 | 22.22 | 6.81 | 111.09 |
| | 40 | 46.78 | 6.59 | 116.95 |
| | 200 | 219.64 | 5.67 | 109.82 |
| | 400 | 453.2 | 4.38 | 113.3 |
| | 1000 | 1024.32 | 6.21 | 102.43 |
| | 2000 | 1893.84 | 2.56 | 94.69 |
| Desipramine | 20 | 20.07 | 3.45 | 100.34 |
| | 40 | 41.77 | 5.62 | 104.44 |
| | 200 | 212.74 | 9.55 | 106.37 |
| | 400 | 437.84 | 4.63 | 109.46 |
| | 1000 | 957.96 | 6.11 | 95.8 |
| | 2000 | 1989.62 | 7.72 | 99.48 |
| Desmethyltramadol | 50 | 37.56 | 9.94 | 75.12 |
| | 100 | 107.72 | 4.86 | 107.72 |
| | 500 | 621.11 | 7.49 | 124.22 |
| | 1000 | 1055.88 | 8.46 | 105.59 |
| | 2500 | 1943.94 | 9.43 | 77.76 |
| | 5000 | 2617.24 | 3.26 | 52.34 |
| Desmethyltramadol | 50 | 43.85 | 7.11 | 87.71 |
| | 100 | 101.33 | 5.15 | 101.33 |
| | 500 | 551.35 | 7.12 | 110.27 |
| | 1000 | 1072.2 | 6.47 | 107.22 |
| | 2500 | 2293.45 | 5.74 | 91.74 |
| | 5000 | 5090.48 | 5.04 | 101.81 |
| Desomorphine | 50 | 59.4 | 6.45 | 118.8 |
| | 100 | 120.71 | 12.89 | 120.71 |
| | 500 | 586.47 | 8.15 | 117.29 |
| | 1000 | 1148.7 | 5.54 | 114.87 |
| | 2500 | 2370.25 | 10.74 | 94.81 |
| | 5000 | 4795.48 | 5.81 | 95.91 |
| DesTapentadol | 50 | 35.32 | 14.29 | 70.64 |
| | 100 | 112.82 | 5.81 | 112.82 |
| | 500 | 601.01 | 5.59 | 120.2 |
| | 1000 | 1083.9 | 5.14 | 108.39 |
| | 2500 | 2057.18 | 6.41 | 82.29 |
| | 5000 | 4946.59 | 2.43 | 98.93 |
| DesTapentadol | 50 | 41.3 | 13.90 | 70.64 |
| | 100 | 128.94 | 5.70 | 112.82 |
| | 500 | 653.56 | 5.28 | 120.2 |
| | 1000 | 1126.01 | 4.59 | 108.39 |
| | 2500 | 1937.11 | 5.04 | 82.29 |
| | 5000 | 3382.97 | 3.65 | 98.93 |
| Dextromethorphan | 50 | 61.12 | 5.61 | 122.24 |
| | 100 | 126.92 | 5.34 | 126.92 |
| | 500 | 625.19 | 8.29 | 123.04 |
| | 1000 | 1248.79 | 2.62 | 124.88 |
| | 2500 | 2591.04 | 3.53 | 103.64 |
| | 5000 | 4425.94 | 5.88 | 88.52 |
| Dextrorphan | 50 | 58.26 | 9.27 | 116.52 |
| | 100 | 110.63 | 5.94 | 110.63 |
| | 500 | 546.6 | 6.57 | 109.32 |
| | 1000 | 1015.62 | 6.46 | 101.56 |
| | 2500 | 2408.01 | 3.79 | 96.32 |
| | 5000 | 5010.87 | 6.20 | 100.22 |
| Diazepam | 10 | 10.02 | 2.31 | 100.17 |
| | 20 | 20.45 | 1.43 | 102.24 |
| | 100 | 99.91 | 2.10 | 99.91 |
| | 200 | 203.53 | 1.46 | 101.76 |
| | 500 | 471.52 | 1.58 | 94.3 |
| | 1000 | 1024.58 | 2.25 | 102.46 |
| Dihydrocodeine | 50 | 49.17 | 5.85 | 98.35 |
| | 100 | 93.29 | 11.17 | 93.29 |
| | 500 | 484.06 | 10.80 | 96.81 |
| | 1000 | 928.81 | 8.43 | 92.88 |
| | 2500 | 2424.79 | 6.71 | 96.99 |
| | 5000 | 5169.87 | 7.31 | 103.4 |
| EDDP | 50 | 51.05 | 2.70 | 102.1 |
| | 100 | 98.72 | 7.24 | 98.72 |
| | 500 | 498.15 | 4.64 | 99.63 |
| | 1000 | 1008.52 | 4.67 | 100.85 |
| | 2500 | 2317.71 | 1.87 | 92.71 |
| | 5000 | 5175.86 | 4.24 | 103.52 |
| Fentanyl | 2 | 1.93 | 8.18 | 96.59 |
| | 4 | 3.95 | 5.60 | 98.75 |
| | 20 | 20 | 5.68 | 100.02 |
| | 40 | 40.16 | 4.36 | 100.41 |
| | 100 | 94.55 | 5.23 | 94.55 |
| | 200 | 205.4 | 4.16 | 102.7 |
| Flunitrazepam | 10 | 9.56 | 12.16 | 95.63 |
| | 20 | 19.37 | 9.53 | 96.87 |
| | 100 | 90.35 | 8.30 | 90.35 |
| | 200 | 188.97 | 7.02 | 94.48 |
| | 500 | 458.9 | 4.13 | 91.78 |
| | 1000 | 1062.85 | 6.59 | 106.28 |
| Gabapentin | 100 | 100.97 | 10.40 | 100.97 |
| | 200 | 186.78 | 5.25 | 93.39 |
| | 1000 | 865.03 | 4.93 | 86.5 |
| | 2000 | 1722.23 | 8.55 | 86.11 |
| | 5000 | 4386.95 | 7.11 | 87.74 |
| | 10000 | 11038.04 | 7.49 | 110.38 |
| Hydrocodone | 50 | 53.97 | 11.54 | 107.95 |
| | 100 | 107.92 | 9.15 | 107.92 |
| | 500 | 537.89 | 5.65 | 107.58 |
| | 1000 | 1066.67 | 8.79 | 106.67 |
| | 2500 | 2439.19 | 6.23 | 97.57 |
| | 5000 | 4944.35 | 7.44 | 98.89 |
| Hydromorphone | 50 | 51.86 | 2.04 | 103.72 |
| | 100 | 99.52 | 2.83 | 99.52 |
| | 500 | 489.68 | 5.05 | 97.94 |
| | 1000 | 1007.19 | 3.02 | 100.72 |
| | 2500 | 2366.96 | 4.67 | 94.68 |
| | 5000 | 5134.79 | 4.53 | 102.7 |
| Imipramine | 20 | 25.18 | 7.20 | 125.9 |
| | 40 | 51.14 | 5.28 | 127.85 |
| | 200 | 247.68 | 5.48 | 123.84 |
| | 400 | 476.31 | 3.29 | 119.08 |
| | 1000 | 983.45 | 4.61 | 98.35 |
| | 2000 | 1876.24 | 5.30 | 93.81 |
| Lorazepam | 10 | 11.6 | 5.96 | 115.95 |
| | 20 | 24.9 | 5.96 | 124.51 |
| | 100 | 109.85 | 6.87 | 109.85 |
| | 200 | 211.98 | 7.68 | 105.99 |
| | 500 | 505.18 | 4.46 | 101.04 |
| | 1000 | 966.5 | 4.17 | 96.65 |
| MDA | 50 | 58.97 | 11.25 | 117.95 |
| | 100 | 113.77 | 12.69 | 113.77 |
| | 500 | 517.22 | 10.24 | 103.44 |
| | 1000 | 1071.89 | 12.18 | 107.19 |
| | 2500 | 2341.24 | 9.74 | 93.65 |
| | 5000 | 5046.91 | 17.14 | 100.94 |
| MDMA | 50 | 53.76 | 5.72 | 107.52 |
| | 100 | 102.58 | 4.91 | 102.58 |
| | 500 | 503.81 | 4.51 | 100.76 |
| | 1000 | 997.64 | 5.81 | 99.76 |
| | 2500 | 2312.37 | 7.09 | 92.49 |
| | 5000 | 5179.83 | 6.16 | 103.6 |
| Meperidine | 50 | 52.23 | 5.13 | 104.45 |
| | 100 | 106.63 | 2.34 | 106.63 |
| | 500 | 519.44 | 2.94 | 103.89 |
| | 1000 | 1028.08 | 3.55 | 102.81 |
| | 2500 | 2377.1 | 5.96 | 95.08 |
| | 5000 | 5066.51 | 5.20 | 101.33 |
| Meprobamate | 50 | 62.32 | 4.42 | 124.64 |
| | 100 | 118.12 | 8.97 | 118.12 |
| | 500 | 621.68 | 7.31 | 124.34 |
| | 1000 | 1249.61 | 7.24 | 124.96 |
| | 2500 | 2460.62 | 7.27 | 98.42 |
| | 5000 | 4637.66 | 7.85 | 92.75 |
| Methadone | 50 | 50.22 | 4.30 | 100.43 |
| | 100 | 104.15 | 2.24 | 104.15 |
| | 500 | 520.93 | 3.14 | 104.19 |
| | 1000 | 1068.53 | 4.35 | 106.85 |
| | 2500 | 2347.98 | 4.24 | 93.92 |
| | 5000 | 5058.21 | 4.40 | 101.16 |
| Methamphetamine | 50 | 53.77 | 6.90 | 107.55 |
| | 100 | 109.99 | 10.11 | 109.99 |
| | 500 | 543.03 | 7.94 | 108.61 |
| | 1000 | 1079.8 | 9.14 | 107.98 |
| | 2500 | 2428.15 | 8.16 | 97.13 |
| | 5000 | 4935.25 | 8.83 | 98.71 |
| Methylphenidate | 50 | 40.65 | 8.36 | 81.3 |
| | 100 | 107.03 | 5.18 | 107.03 |
| | 500 | 577.36 | 3.23 | 115.47 |
| | 1000 | 1036.55 | 3.53 | 103.66 |
| | 2500 | 2214.58 | 8.68 | 88.58 |
| | 5000 | 5042.33 | 4.89 | 100.85 |
| Midazolam | 10 | 7.76 | 10.12 | 77.57 |
| | 20 | 15.35 | 2.68 | 76.75 |
| | 100 | 75.27 | 6.96 | 75.27 |
| | 200 | 168.36 | 6.37 | 84.18 |
| | 500 | 448.16 | 3.93 | 89.63 |
| | 1000 | 1115.1 | 6.18 | 111.51 |
| Morphine | 50 | 52.04 | 4.79 | 104.08 |
| | 100 | 103.13 | 3.00 | 103.13 |
| | 500 | 475.86 | 4.15 | 95.17 |
| | 1000 | 971.74 | 3.47 | 97.17 |
| | 2500 | 2314.21 | 2.14 | 92.57 |
| | 5000 | 5233.01 | 2.22 | 104.66 |
| Naloxone | 10 | 11.01 | 7.79 | 110.06 |
| | 20 | 22.14 | 8.03 | 110.69 |
| | 100 | 105.01 | 6.49 | 105.01 |
| | 200 | 201.16 | 8.25 | 100.58 |
| | 500 | 494.8 | 6.74 | 98.96 |
| | 1000 | 995.88 | 10.46 | 99.59 |
| Norbuprenorphine | 10 | 9.62 | 11.12 | 96.17 |
| | 20 | 21.54 | 12.20 | 107.71 |
| | 100 | 96.04 | 5.48 | 96.04 |
| | 200 | 192.75 | 11.12 | 96.37 |
| | 500 | 467.43 | 7.99 | 93.49 |
| | 1000 | 1042.63 | 8.47 | 104.26 |
| Nordiazepam | 10 | 9.53 | 3.44 | 95.29 |
| | 20 | 18.75 | 3.95 | 93.74 |
| | 100 | 92.96 | 3.57 | 92.96 |
| | 200 | 190.63 | 1.97 | 95.31 |
| | 500 | 454.01 | 3.03 | 90.8 |
| | 1000 | 1064.13 | 1.94 | 106.41 |
| Norfentanyl | 2 | 2.49 | 6.82 | 124.62 |
| | 4 | 4.78 | 6.59 | 119.42 |
| | 20 | 22.2 | 4.35 | 111.01 |
| | 40 | 43.69 | 4.30 | 109.23 |
| | 100 | 94.56 | 4.76 | 94.56 |
| | 200 | 198.28 | 5.72 | 99.14 |
| Normeperidine | 50 | 40.77 | 10.36 | 81.53 |
| | 100 | 103.81 | 8.14 | 103.81 |
| | 500 | 584.21 | 4.72 | 116.84 |
| | 1000 | 1080.51 | 8.67 | 108.05 |
| | 2500 | 2132.03 | 6.69 | 85.28 |
| | 5000 | 4983.46 | 11.42 | 99.67 |
| Norpropoxyphene | 50 | 51.15 | 8.66 | 102.29 |
| | 100 | 115.47 | 6.79 | 115.47 |
| | 500 | 529.06 | 7.83 | 105.81 |
| | 1000 | 1120.26 | 6.56 | 112.03 |
| | 2500 | 2482.96 | 6.33 | 99.32 |
| | 5000 | 4851.11 | 5.91 | 97.02 |
| Nortriptyline | 20 | 20.79 | 5.43 | 103.96 |
| | 40 | 43.25 | 9.28 | 108.12 |
| | 200 | 203.92 | 7.45 | 101.96 |
| | 400 | 437.37 | 4.05 | 109.34 |
| | 1000 | 1002.25 | 6.73 | 100.22 |
| | 2000 | 1952.43 | 3.75 | 97.62 |
| Oxazepam | 10 | 9.94 | 7.25 | 99.42 |
| | 20 | 18.96 | 6.43 | 94.8 |
| | 100 | 91.3 | 6.77 | 91.3 |
| | 200 | 195 | 7.77 | 97.5 |
| | 500 | 443.4 | 5.30 | 88.68 |
| | 1000 | 1071.41 | 7.58 | 107.14 |
| Oxycodone | 50 | 59.12 | 5.59 | 118.24 |
| | 100 | 112.03 | 5.90 | 112.03 |
| | 500 | 521.75 | 7.96 | 104.35 |
| | 1000 | 1082.02 | 6.71 | 108.2 |
| | 2500 | 2438.31 | 4.37 | 97.53 |
| | 5000 | 4936.78 | 4.39 | 98.74 |
| Oxymorphone | 50 | 49.56 | 2.72 | 99.11 |
| | 100 | 97.74 | 2.61 | 97.74 |
| | 500 | 487.94 | 2.84 | 97.59 |
| | 1000 | 1014 | 2.59 | 101.4 |
| | 2500 | 2298.61 | 3.44 | 91.94 |
| | 5000 | 5202.15 | 3.48 | 104.04 |
| PCP | 4 | 4.47 | 5.98 | 111.71 |
| | 8 | 8.54 | 6.85 | 106.78 |
| | 40 | 40.16 | 6.95 | 100.39 |
| | 80 | 82.5 | 6.07 | 103.12 |
| | 200 | 189.19 | 6.55 | 94.59 |
| | 400 | 407.15 | 7.34 | 101.79 |
| Pregabalin | 50 | 37.8 | 16.92 | 75.59 |
| | 100 | 109.4 | 6.68 | 109.4 |
| | 500 | 588.84 | 6.08 | 117.77 |
| | 1000 | 1076.22 | 9.81 | 107.62 |
| | 2500 | 2135.92 | 9.65 | 85.44 |
| | 5000 | 5134.68 | 14.35 | 102.69 |
| Pregabalin | 50 | 51.89 | 4.47 | 103.78 |
| | 100 | 103.88 | 8.33 | 103.88 |
| | 500 | 500.98 | 5.71 | 100.2 |
| | 1000 | 959.36 | 7.37 | 95.94 |
| | 2500 | 2276.43 | 6.31 | 91.06 |
| | 5000 | 5257.47 | 6.72 | 105.15 |
| Propoxyphene | 50 | 47.89 | 11.61 | 95.78 |
| | 100 | 102.21 | 11.30 | 102.21 |
| | 500 | 520.52 | 7.66 | 104.1 |
| | 1000 | 1011.32 | 13.48 | 101.13 |
| | 2500 | 2382.82 | 13.76 | 95.31 |
| | 5000 | 5085.24 | 11.02 | 101.7 |
| Ritalinic Acid | 100 | 120.21 | 7.20 | 120.21 |
| | 200 | 246.88 | 6.43 | 123.44 |
| | 1000 | 1287.35 | 4.06 | 128.73 |
| | 2000 | 2080.99 | 4.51 | 104.05 |
| | 5000 | 4056.9 | 7.55 | 81.14 |
| | 10000 | 9839.57 | 8.62 | 98.4 |
| Sertraline | 20 | 16.85 | 4.59 | 84.27 |
| | 40 | 33.39 | 4.82 | 83.47 |
| | 200 | 175.01 | 5.65 | 87.5 |
| | 400 | 393.38 | 4.32 | 98.34 |
| | 1000 | 986.91 | 4.68 | 98.69 |
| | 2000 | 2054.47 | 4.69 | 102.72 |
| Tapentadol | 50 | 62.65 | 5.69 | 125.3 |
| | 100 | 128.26 | 5.05 | 128.26 |
| | 500 | 569.26 | 4.01 | 113.85 |
| | 1000 | 1236.2 | 5.74 | 123.62 |
| | 2500 | 2377.24 | 3.49 | 95.09 |
| | 5000 | 4644.8 | 4.01 | 92.9 |
| Temazepam | 10 | 8.35 | 7.78 | 83.49 |
| | 20 | 16.84 | 4.55 | 84.22 |
| | 100 | 84.3 | 7.19 | 84.3 |
| | 200 | 180.92 | 6.95 | 90.46 |
| | 500 | 458.11 | 3.71 | 91.62 |
| | 1000 | 1081.48 | 5.70 | 108.15 |
| Tramadol | 50 | 44.36 | 9.82 | 88.72 |
| | 100 | 103.96 | 6.97 | 103.96 |
| | 500 | 538.48 | 9.18 | 107.7 |
| | 1000 | 1059.56 | 7.72 | 105.96 |
| | 2500 | 2257.6 | 9.31 | 90.3 |
| | 5000 | 4634.21 | 9.15 | 92.68 |
| Triazolam | 10 | 7.37 | 6.93 | 73.68 |
| | 20 | 14.3 | 5.29 | 71.51 |
| | 100 | 71.96 | 8.60 | 71.96 |
| | 200 | 160.84 | 9.39 | 80.42 |
| | 500 | 439.54 | 2.26 | 87.91 |
| | 1000 | 1135.99 | 6.28 | 113.6 |
| Zaleplon | 10 | 12.21 | 11.15 | 122.1 |
| | 20 | 24.61 | 7.91 | 123.05 |
| | 100 | 123.78 | 7.38 | 123.78 |
| | 200 | 237.4 | 8.23 | 118.7 |
| | 500 | 497.49 | 6.71 | 99.5 |
| | 1000 | 929 | 5.86 | 92.9 |
| Zopiclone | 10 | 7.92 | 12.50 | 79.2 |
| | 20 | 25.06 | 7.91 | 125.31 |
| | 100 | 116.69 | 9.29 | 116.69 |
| | 200 | 177.16 | 7.16 | 88.58 |
| | 500 | 435.26 | 8.00 | 87.05 |
| | 1000 | 945.84 | 10.46 | 94.58 |
| Zolpidem | 10 | 10.28 | 7.07 | 102.8 |
| | 20 | 20.24 | 5.94 | 101.19 |
| | 100 | 100.19 | 4.11 | 100.19 |
| | 200 | 200.89 | 4.50 | 100.45 |
| | 500 | 468.89 | 3.97 | 93.78 |
| | 1000 | 1029.52 | 2.76 | 102.95 |
| Carboxy Zolpidem | 10 | 12.27 | 6.01 | 122.7 |
| | 20 | 23.29 | 3.49 | 116.45 |
| | 100 | 123.21 | 4.89 | 123.21 |
| | 200 | 237.56 | 5.47 | 118.78 |
| | 500 | 481.45 | 4.84 | 96.29 |
| | 1000 | 946.15 | 6.97 | 94.62 |
| Butalbital | 50 | 50.08 | 7.48 | 100.15 |
| | 100 | 100.86 | 10.56 | 100.86 |
| | 500 | 465.91 | 5.93 | 93.18 |
| | 1000 | 1000.05 | 5.67 | 100.01 |
| | 2500 | 2328.06 | 7.40 | 93.12 |
| | 5000 | 5205.04 | 6.02 | 104.1 |
| THC | 10 | 10.78 | 4.94 | 107.77 |
| | 20 | 17.65 | 4.14 | 88.26 |
| | 100 | 81.09 | 3.81 | 81.09 |
| | 200 | 184.76 | 2.33 | 92.38 |
| | 500 | 533.47 | 1.30 | 106.69 |
| | 1000 | 1238.05 | 1.70 | 123.8 |
| Phenobarbital | 50 | 52.37 | 6.79 | 104.73 |
| | 100 | 99.61 | 5.91 | 99.61 |
| | 500 | 490.34 | 5.79 | 98.07 |
| | 1000 | 971.15 | 3.90 | 97.12 |
| | 2500 | 2253.82 | 4.60 | 90.15 |
| | 5000 | 5282.71 | 5.42 | 105.65 |

TABLE 10
Inter-day accuracy and precision studied for 7 runs

| | Actual | Mean | CV | |
|---|---|---|---|---|
| Component Name | (ng/mL) | n = 7 | (%) | Accuracy |
| 6-MAM | 2 | 1.93 | 12.20 | 96.5 |
| | 4 | 3.616 | 8.61 | 90.4 |
| | 20 | 19.019 | 7.92 | 95.095 |
| | 40 | 40.122 | 6.88 | 100.305 |
| | 100 | 95.868 | 7.69 | 95.868 |
| | 200 | 205.445 | 7.63 | 102.7225 |
| OH-et-Flurazepam | 10 | 9.145 | 12.89 | 91.45 |
| | 20 | 18.462 | 5.00 | 92.31 |
| | 100 | 96.227 | 1.89 | 96.227 |
| | 200 | 197.936 | 2.86 | 98.968 |
| | 500 | 480.16 | 4.42 | 96.032 |
| | 1000 | 1028.071 | 9.00 | 102.8071 |
| 4-Hydroxyalprazolam | 10 | 10.293 | 7.62 | 102.93 |
| | 20 | 20.108 | 4.27 | 100.54 |
| | 100 | 100.205 | 6.87 | 100.205 |
| | 200 | 209.968 | 6.08 | 104.984 |
| | 500 | 482.232 | 3.58 | 96.4464 |
| | 1000 | 1007.194 | 9.72 | 100.7194 |
| 7-Aminoclonazepam | 10 | 9.83 | 13.03 | 98.3 |
| | 20 | 19.096 | 8.23 | 95.48 |
| | 100 | 93.704 | 5.67 | 93.704 |
| | 200 | 197.896 | 9.11 | 98.948 |
| | 500 | 485.943 | 9.47 | 97.1886 |
| | 1000 | 1023.531 | 14.16 | 102.3531 |
| Amitriptyline | 20 | 21.327 | 9.34 | 106.635 |
| | 40 | 40.661 | 3.10 | 101.6525 |
| | 200 | 199.041 | 7.35 | 99.5205 |
| | 400 | 415.941 | 3.17 | 103.98525 |
| | 1000 | 998.277 | 6.12 | 99.8277 |
| | 2000 | 1984.752 | 8.55 | 99.2376 |
| Benzoylecgonine | 50 | 59.513 | 6.31 | 119.026 |
| | 100 | 115.159 | 6.75 | 115.159 |
| | 500 | 579.174 | 5.71 | 115.8348 |
| | 1000 | 1124.089 | 5.01 | 112.4089 |
| | 2500 | 2558.48 | 4.66 | 102.3392 |
| | 5000 | 4713.587 | 5.44 | 94.27174 |
| Buprenorphine | 5 | 5.143 | 12.30 | 102.86 |
| | 10 | 9.647 | 12.18 | 96.47 |
| | 50 | 47.809 | 11.55 | 95.618 |
| | 100 | 95.13 | 5.13 | 95.13 |
| | 250 | 252.313 | 4.66 | 100.9252 |
| | 500 | 504.958 | 10.12 | 100.9916 |
| Carisoprodol | 50 | 59.774 | 9.38 | 119.548 |
| | 100 | 116.419 | 10.01 | 116.419 |
| | 500 | 574.089 | 8.19 | 114.8178 |
| | 1000 | 1172.398 | 6.04 | 117.2398 |
| | 2500 | 2513.025 | 11.58 | 100.521 |
| | 5000 | 4714.295 | 13.34 | 94.2859 |
| Chlordiazepoxide | 10 | 9.379 | 11.18 | 93.79 |
| | 20 | 17.617 | 11.81 | 88.085 |
| | 100 | 90.256 | 11.18 | 90.256 |
| | 200 | 201.645 | 11.43 | 100.8225 |
| | 500 | 511.167 | 13.46 | 102.2334 |
| | 1000 | 999.889 | 10.34 | 99.9889 |
| Codeine | 50 | 49.854 | 8.63 | 99.708 |
| | 100 | 95.894 | 9.67 | 95.894 |
| | 500 | 508.453 | 8.69 | 101.6906 |
| | 1000 | 1011.263 | 9.92 | 101.1263 |
| | 2500 | 2380.853 | 10.16 | 95.23412 |
| | 5000 | 5103.683 | 9.48 | 102.07366 |
| Cyclobenzaprine | 20 | 23.485 | 13.01 | 117.425 |
| | 40 | 44.509 | 5.12 | 111.2725 |
| | 200 | 221.073 | 7.97 | 110.5365 |
| | 400 | 442.333 | 4.72 | 110.58325 |
| | 1000 | 998.628 | 4.61 | 99.8628 |
| | 2000 | 1929.973 | 7.26 | 96.49865 |
| Desipramine | 20 | 21.342 | 5.85 | 106.71 |
| | 40 | 40.636 | 4.64 | 101.59 |
| | 200 | 223.338 | 4.15 | 111.669 |
| | 400 | 430.5 | 6.55 | 107.625 |
| | 1000 | 1013.344 | 5.12 | 101.3344 |
| | 2000 | 1930.84 | 6.49 | 96.542 |
| Desmethyltramadol | 50 | 39.342 | 7.94 | 78.684 |
| | 100 | 105.554 | 11.12 | 105.554 |
| | 500 | 603.255 | 9.63 | 120.651 |
| | 1000 | 1073.545 | 10.42 | 107.3545 |
| | 2500 | 1985.786 | 9.14 | 79.43144 |
| | 5000 | 4714.284 | 0.58 | 94.28568 |
| Desomorphine | 50 | 58.837 | 12.17 | 117.674 |
| | 100 | 127.902 | 10.65 | 127.902 |
| | 500 | 616.638 | 8.90 | 123.3276 |
| | 1000 | 1139.192 | 6.40 | 113.9192 |
| | 2500 | 2380.669 | 10.36 | 95.22676 |
| | 5000 | 4816.762 | 9.79 | 96.33524 |
| DesTapentadol | 50 | 39.3 | 9.19 | 78.6 |
| | 100 | 106.631 | 9.00 | 106.631 |
| | 500 | 589.536 | 5.96 | 117.9072 |
| | 1000 | 1074.349 | 9.62 | 107.4349 |
| | 2500 | 2108.364 | 9.17 | 84.33456 |
| | 5000 | 4938.339 | 2.33 | 98.76678 |
| Dextromethorphan | 50 | 57.101 | 8.00 | 114.202 |
| | 100 | 126.855 | 5.70 | 126.855 |
| | 500 | 567.615 | 8.43 | 113.523 |
| | 1000 | 1260.622 | 7.32 | 126.0622 |
| | 2500 | 2557.931 | 8.40 | 102.31724 |
| | 5000 | 4459.877 | 8.10 | 89.19754 |
| Dextrorphan | 50 | 60.202 | 13.50 | 120.404 |
| | 100 | 119.585 | 9.20 | 119.585 |
| | 500 | 565.957 | 6.99 | 113.1914 |
| | 1000 | 1062.165 | 14.11 | 106.2165 |
| | 2500 | 2534.949 | 11.67 | 101.39796 |
| | 5000 | 4807.142 | 8.92 | 96.14284 |
| Diazepam | 10 | 10.335 | 6.02 | 103.35 |
| | 20 | 20.196 | 3.58 | 100.98 |
| | 100 | 100.989 | 4.50 | 100.989 |
| | 200 | 205.221 | 2.82 | 102.6105 |
| | 500 | 496.036 | 2.17 | 99.2072 |
| | 1000 | 997.223 | 5.70 | 99.7223 |
| Dihydrocodeine | 50 | 50.689 | 6.86 | 101.378 |
| | 100 | 96.589 | 5.67 | 96.589 |
| | 500 | 518.144 | 9.90 | 103.6288 |
| | 1000 | 1031.237 | 9.39 | 103.1237 |
| | 2500 | 2384.294 | 9.75 | 95.37176 |
| | 5000 | 5069.047 | 10.55 | 101.38094 |
| EDDP | 50 | 52.373 | 9.29 | 104.746 |
| | 100 | 104.689 | 5.74 | 104.689 |
| | 500 | 513.104 | 7.34 | 102.6208 |
| | 1000 | 1034.748 | 7.05 | 103.4748 |
| | 2500 | 2502.021 | 7.85 | 100.08084 |
| | 5000 | 4943.064 | 8.43 | 98.86128 |
| Fentanyl | 2 | 2.102 | 6.68 | 105.1 |
| | 4 | 3.959 | 4.65 | 98.975 |
| | 20 | 19.736 | 6.07 | 98.68 |
| | 40 | 40.809 | 5.15 | 102.0225 |
| | 100 | 99.57 | 7.53 | 99.57 |
| | 200 | 199.824 | 5.01 | 99.912 |
| Flunitrazepam | 10 | 10.279 | 12.47 | 102.79 |
| | 20 | 20.037 | 9.02 | 100.185 |
| | 100 | 89.661 | 14.99 | 89.661 |
| | 200 | 191.934 | 12.91 | 95.967 |
| | 500 | 469.029 | 12.40 | 93.8058 |
| | 1000 | 1068.684 | 7.77 | 106.8684 |
| Gabapentin | 100 | 113.582 | 7.63 | 113.582 |
| | 200 | 210.796 | 11.85 | 105.398 |
| | 1000 | 1001.313 | 8.49 | 100.1313 |
| | 2000 | 1938.305 | 5.77 | 96.91525 |
| | 5000 | 4793.645 | 14.25 | 95.8729 |
| | 10000 | 10242.359 | 11.87 | 102.42359 |
| Hydrocodone | 50 | 56.75 | 8.27 | 113.5 |
| | 100 | 108.487 | 6.51 | 108.487 |
| | 500 | 535.285 | 7.52 | 107.057 |
| | 1000 | 1076.554 | 4.47 | 107.6554 |
| | 2500 | 2391.087 | 6.39 | 95.64348 |
| | 5000 | 4981.836 | 7.59 | 99.63672 |
| Hydromorphone | 50 | 50.036 | 5.29 | 100.072 |
| | 100 | 101.853 | 3.52 | 101.853 |
| | 500 | 497.046 | 3.97 | 99.4092 |
| | 1000 | 1015.242 | 3.44 | 101.5242 |
| | 2500 | 2409.174 | 5.04 | 96.36696 |
| | 5000 | 5076.649 | 7.21 | 101.53298 |
| Imipramine | 20 | 25.249 | 6.92 | 126.245 |
| | 40 | 49.328 | 7.01 | 123.32 |
| | 200 | 245.662 | 3.32 | 122.831 |
| | 400 | 481.357 | 4.11 | 120.33925 |
| | 1000 | 1014.236 | 7.43 | 101.4236 |
| | 2000 | 1844.167 | 5.63 | 92.20835 |
| Lorazepam | 10 | 12.221 | 2.68 | 122.21 |
| | 20 | 23.327 | 3.93 | 116.635 |
| | 100 | 110.648 | 7.02 | 110.648 |
| | 200 | 223.622 | 7.19 | 111.811 |
| | 500 | 524.027 | 5.76 | 104.8054 |
| | 1000 | 936.155 | 7.19 | 93.6155 |
| MDA | 50 | 56.424 | 10.54 | 112.848 |
| | 100 | 115.115 | 14.11 | 115.115 |
| | 500 | 577.012 | 12.89 | 115.4024 |
| | 1000 | 1027.623 | 10.10 | 102.7623 |
| | 2500 | 2415.519 | 7.28 | 96.62076 |
| | 5000 | 4958.307 | 9.97 | 99.16614 |
| MDMA | 50 | 60.24 | 17.77 | 120.48 |
| | 100 | 112.654 | 11.58 | 112.654 |
| | 500 | 529.626 | 14.07 | 105.9252 |
| | 1000 | 1044.036 | 11.34 | 104.4036 |
| | 2500 | 2406.061 | 8.12 | 96.24244 |
| | 5000 | 4997.382 | 10.02 | 99.94764 |
| Meperidine | 50 | 54.096 | 5.96 | 108.192 |
| | 100 | 106.027 | 4.57 | 106.027 |
| | 500 | 531.724 | 5.46 | 106.3448 |
| | 1000 | 1050.335 | 4.88 | 105.0335 |
| | 2500 | 2494.327 | 7.30 | 99.77308 |
| | 5000 | 4913.491 | 7.53 | 98.26982 |
| Meprobamate | 50 | 58.591 | 13.27 | 117.182 |
| | 100 | 124.163 | 12.01 | 124.163 |
| | 500 | 620.061 | 9.60 | 124.0122 |
| | 1000 | 1198.158 | 7.49 | 119.8158 |
| | 2500 | 2510.349 | 7.29 | 100.41396 |
| | 5000 | 4628.679 | 5.61 | 92.57358 |
| Methadone | 50 | 51.255 | 3.85 | 102.51 |
| | 100 | 102.709 | 4.03 | 102.709 |
| | 500 | 522.547 | 4.66 | 104.5094 |
| | 1000 | 1048.074 | 4.51 | 104.8074 |
| | 2500 | 2470.304 | 2.87 | 98.81216 |
| | 5000 | 4955.111 | 2.91 | 99.10222 |
| Methamphetamine | 50 | 58.358 | 10.68 | 116.716 |
| | 100 | 110.745 | 7.70 | 110.745 |
| | 500 | 558.027 | 11.54 | 111.6054 |
| | 1000 | 1107.356 | 6.84 | 110.7356 |
| | 2500 | 2496.679 | 6.06 | 99.86716 |
| | 5000 | 4818.835 | 7.39 | 96.3767 |
| Methylphenidate | 50 | 41.532 | 7.54 | 83.064 |
| | 100 | 78.541 | 5.94 | 78.541 |
| | 500 | 419.333 | 4.81 | 83.8666 |
| | 1000 | 1236.24 | 7.47 | 123.624 |
| | 2500 | 2786.336 | 5.60 | 111.45344 |
| | 5000 | 4629.931 | 11.54 | 92.59862 |
| Midazolam | 10 | 8.281 | 8.96 | 82.81 |
| | 20 | 16.408 | 5.54 | 82.04 |
| | 100 | 84.021 | 9.19 | 84.021 |
| | 200 | 180.579 | 9.69 | 90.2895 |
| | 500 | 477.496 | 7.69 | 95.4992 |
| | 1000 | 1063.214 | 13.88 | 106.3214 |
| Morphine | 50 | 51.924 | 5.18 | 103.848 |
| | 100 | 102.435 | 5.23 | 102.435 |
| | 500 | 501.113 | 3.63 | 100.2226 |
| | 1000 | 1008.631 | 1.84 | 100.8631 |
| | 2500 | 2437.596 | 3.44 | 97.50384 |
| | 5000 | 5048.301 | 6.24 | 100.96602 |
| Naloxone | 10 | 11.592 | 8.74 | 115.92 |
| | 20 | 22.361 | 9.36 | 111.805 |
| | 100 | 109.608 | 6.13 | 109.608 |
| | 200 | 226.355 | 5.99 | 113.1775 |
| | 500 | 483.011 | 4.40 | 96.6022 |
| | 1000 | 977.073 | 11.87 | 97.7073 |
| Norbuprenorphine | 10 | 9.619 | 19.45 | 96.19 |
| | 20 | 21.224 | 12.12 | 106.12 |
| | 100 | 107.124 | 4.89 | 107.124 |
| | 200 | 198.969 | 7.87 | 99.4845 |
| | 500 | 484.094 | 8.81 | 96.8188 |
| | 1000 | 1008.97 | 6.72 | 100.897 |
| Nordiazepam | 10 | 9.635 | 10.50 | 96.35 |
| | 20 | 19.99 | 6.79 | 99.95 |
| | 100 | 98.472 | 4.63 | 98.472 |
| | 200 | 195.575 | 4.81 | 97.7875 |
| | 500 | 486.519 | 5.25 | 97.3038 |
| | 1000 | 1019.809 | 5.67 | 101.9809 |
| Norfentanyl | 2 | 2.15 | 4.98 | 107.5 |
| | 4 | 4.177 | 4.81 | 104.425 |
| | 20 | 21.008 | 4.66 | 105.04 |
| | 40 | 41.422 | 9.35 | 103.555 |
| | 100 | 100.387 | 14.25 | 100.387 |
| | 200 | 196.856 | 14.93 | 98.428 |
| Normeperidine | 50 | 62.319 | 4.03 | 124.638 |
| | 100 | 126.811 | 9.47 | 126.811 |
| | 500 | 642.542 | 5.69 | 128.5084 |
| | 1000 | 1200.314 | 7.22 | 120.0314 |
| | 2500 | 2724.204 | 10.19 | 108.96816 |
| | 5000 | 4651.934 | 7.82 | 93.03868 |
| Norpropoxyphene | 50 | 57.505 | 11.63 | 115.01 |
| | 100 | 106.874 | 8.33 | 106.874 |
| | 500 | 569.853 | 8.41 | 113.9706 |
| | 1000 | 1075.36 | 6.76 | 107.536 |
| | 2500 | 2491.723 | 7.10 | 99.66892 |
| | 5000 | 4848.685 | 10.49 | 96.9737 |
| Nortriptyline | 20 | 20.265 | 7.54 | 101.325 |
| | 40 | 41.048 | 4.33 | 102.62 |
| | 200 | 207.141 | 4.25 | 103.5705 |
| | 400 | 430.264 | 3.23 | 107.566 |
| | 1000 | 1001.463 | 5.20 | 100.1463 |
| | 2000 | 1959.819 | 7.62 | 97.99095 |
| Oxazepam | 10 | 10.312 | 12.48 | 103.12 |
| | 20 | 19.77 | 13.90 | 98.85 |
| | 100 | 95.562 | 13.26 | 95.562 |
| | 200 | 206.874 | 9.05 | 103.437 |
| | 500 | 479.802 | 6.71 | 95.9604 |
| | 1000 | 1017.68 | 8.78 | 101.768 |
| Oxycodone | 50 | 57.266 | 7.12 | 114.532 |
| | 100 | 114.272 | 6.37 | 114.272 |
| | 500 | 523.065 | 7.15 | 104.613 |
| | 1000 | 1086.639 | 8.36 | 108.6639 |
| | 2500 | 2466.262 | 5.38 | 98.65048 |
| | 5000 | 4902.496 | 7.86 | 98.04992 |
| Oxymorphone | 50 | 51.45 | 6.20 | 102.9 |
| | 100 | 102.58 | 3.63 | 102.58 |
| | 500 | 525.212 | 4.06 | 105.0424 |
| | 1000 | 1023.084 | 3.34 | 102.3084 |
| | 2500 | 2483.991 | 3.39 | 99.35964 |
| | 5000 | 4963.683 | 6.83 | 99.27366 |
| PCP | 4 | 4.326 | 6.14 | 108.15 |
| | 8 | 8.389 | 10.13 | 104.8625 |
| | 40 | 41.353 | 7.14 | 103.3825 |
| | 80 | 83.883 | 7.82 | 104.85375 |
| | 200 | 189.042 | 6.93 | 94.521 |
| | 400 | 405.007 | 7.71 | 101.25175 |
| Pregabalin | 50 | 57.604 | 13.24 | 115.208 |
| | 100 | 123.443 | 9.64 | 123.443 |
| | 500 | 638.824 | 5.52 | 127.7648 |
| | 1000 | 1232.32 | 4.73 | 123.232 |
| | 2500 | 2485.475 | 6.78 | 99.419 |
| | 5000 | 4352.334 | 9.06 | 87.04668 |
| Propoxyphene | 50 | 56.678 | 10.60 | 113.356 |
| | 100 | 104.987 | 14.21 | 104.987 |
| | 500 | 505.595 | 14.65 | 101.119 |
| | 1000 | 1064.326 | 11.32 | 106.4326 |
| | 2500 | 2450.388 | 13.37 | 98.01552 |
| | 5000 | 4968.026 | 9.85 | 99.36052 |
| Sertraline | 20 | 14.54 | 11.51 | 72.7 |
| | 40 | 29.663 | 14.13 | 74.1575 |
| | 200 | 177.291 | 9.21 | 88.6455 |
| | 400 | 403.396 | 6.10 | 100.849 |
| | 1000 | 1023.69 | 5.85 | 102.369 |
| | 2000 | 2011.421 | 6.22 | 100.57105 |
| Triazolam | 10 | 7.666 | 11.27 | 76.66 |
| | 20 | 14.838 | 6.84 | 74.19 |
| | 100 | 79.783 | 9.45 | 79.783 |
| | 200 | 181.93 | 13.79 | 90.965 |
| | 500 | 468.668 | 11.18 | 93.7336 |
| | 1000 | 1077.115 | 14.49 | 107.7115 |
| Zaleplon | 10 | 11.648 | 8.28 | 116.48 |
| | 20 | 25.18 | 6.51 | 125.9 |
| | 100 | 120.886 | 6.67 | 120.886 |
| | 200 | 230.422 | 5.85 | 115.211 |
| | 500 | 522.275 | 14.49 | 104.455 |
| | 1000 | 917.59 | 9.22 | 91.759 |
| Zolpidem | 10 | 10.411 | 5.29 | 104.11 |
| | 20 | 20.82 | 4.36 | 104.1 |
| | 100 | 103.137 | 6.28 | 103.137 |
| | 200 | 203.121 | 8.46 | 101.5605 |
| | 500 | 502.471 | 4.36 | 100.4942 |
| | 1000 | 990.041 | 7.50 | 99.0041 |
| Carboxy Zolpidem | 10 | 12.582 | 4.76 | 125.82 |
| | 20 | 22.923 | 3.01 | 114.615 |
| | 100 | 123.592 | 7.91 | 123.592 |
| | 200 | 231.672 | 5.30 | 115.836 |
| | 500 | 504.777 | 11.98 | 100.9554 |
| | 1000 | 927.454 | 8.08 | 92.7454 |
| Carisoprodol 1 | 50 | 59.281 | 8.26 | 118.562 |
| | 100 | 114.222 | 9.91 | 114.222 |
| | 500 | 576.377 | 11.83 | 115.2754 |
| | 1000 | 1169.596 | 10.68 | 116.9596 |
| | 2500 | 2580.468 | 12.88 | 103.21872 |
| | 5000 | 4650.057 | 12.81 | 93.00114 |
| Amphetamine 1 | 50 | 64.646 | 10.52 | 129.292 |
| | 100 | 125.28 | 8.88 | 125.28 |
| | 500 | 609.03 | 11.52 | 121.806 |
| | 1000 | 1136.826 | 9.09 | 113.6826 |
| | 2500 | 2402.718 | 10.79 | 96.10872 |
| | 5000 | 4791.499 | 12.45 | 95.82998 |
| Tapentadol 1 | 10 | 8.069 | 9.81 | 80.69 |
| | 20 | 15.677 | 5.65 | 78.385 |
| | 100 | 84.309 | 9.47 | 84.309 |
| | 200 | 179.573 | 9.13 | 89.7865 |
| | 500 | 478.155 | 9.31 | 95.631 |
| | 1000 | 1064.217 | 12.45 | 106.4217 |
| Tramadol 1 | 50 | 58.769 | 13.72 | 117.538 |
| | 100 | 110.987 | 6.24 | 110.987 |
| | 500 | 538.282 | 13.75 | 107.6564 |
| | 1000 | 1097.215 | 12.08 | 109.7215 |
| | 2500 | 2477.924 | 9.88 | 99.11696 |
| | 5000 | 4866.823 | 9.38 | 97.33646 |
| Temazepam 1 | 10 | 7.666 | 10.67 | 76.66 |
| | 20 | 14.838 | 5.81 | 74.19 |
| | 100 | 79.783 | 9.41 | 79.783 |
| | 200 | 181.93 | 12.61 | 90.965 |
| | 500 | 468.668 | 10.79 | 93.7336 |
| | 1000 | 1077.115 | 14.37 | 107.7115 |
| Zopiclone 1 | 10 | 10.411 | 5.52 | 104.11 |
| | 20 | 20.82 | 7 | 104.1 |
| | 100 | 103.137 | 7.84 | 103.137 |
| | 200 | 203.121 | 8.2 | 101.5605 |
| | 500 | 502.471 | 7.26 | 100.4942 |
| | 1000 | 990.041 | 8.48 | 99.0041 |
| Butalbital 1 | 50 | 56.133 | 10.86 | 112.266 |
| | 100 | 106.332 | 8.65 | 106.332 |
| | 500 | 547.55 | 8.61 | 109.51 |
| | 1000 | 1061.016 | 5.75 | 106.1016 |
| | 2500 | 2462.409 | 6.38 | 98.49636 |
| | 5000 | 4916.56 | 10.12 | 98.3312 |
| THCA 1 | 10 | 11.582 | 4.67 | 115.82 |
| | 20 | 22.923 | 5.56 | 114.615 |
| | 100 | 123.592 | 7.19 | 123.592 |
| | 200 | 231.672 | 5.3 | 115.836 |
| | 500 | 504.777 | 10.89 | 100.9554 |
| | 1000 | 927.454 | 7.4 | 92.7454 |
| Ritalinic acid 1 | 50 | 52.594 | 5.18 | 105.188 |
| | 100 | 100.343 | 5.23 | 100.343 |
| | 500 | 509.723 | 3.63 | 101.9446 |
| | 1000 | 1018.434 | 1.84 | 101.8434 |
| | 2500 | 2468.733 | 3.44 | 98.74932 |
| | 5000 | 5000.174 | 6.24 | 100.00348 |
| Phenobarbital 1 | 50 | 51.306 | 3.61 | 102.612 |
| | 100 | 98.075 | 5.50 | 98.075 |
| | 500 | 501.966 | 2.96 | 100.3932 |
| | 1000 | 988.116 | 1.72 | 98.8116 |
| | 2500 | 2468.619 | 2.22 | 98.74476 |
| | 5000 | 5041.918 | 7.36 | 100.83836 |

### Recovery

Recoveries were calculated by adding known concentrations of drug metabolites, to 3 different samples previously analyzed, and then the final concentrations were measured in duplicate. Results were measured as differences between the measured and the theoretical values and expressed as percentage of recovery.

TABLE 11
Intra-day study of recovery with the quality
controls and their yield (n = 10)

| | | Mean | CV |
|---|---|---|---|
| Component Name | QC Name | n = 10 | (%) |
| 6-MAM | C3 | 9.32 | 8.59 |
| OH-et-Flurazepam | BZ 100 | 79.38 | 4.68 |
| 4-Hydroxyalprazolam | BZ 100 | 83.49 | 4.98 |
| 7-Aminoclonazepam | BZ 100 | 112.24 | 6.68 |
| Amitriptyline | POS | 138.38 | 3.54 |
| Amphetamine | POS | 1567.18 | 5.22 |
| Benzoylecgonine | C3 | 196.79 | 2.91 |
| Buprenorphine | PM 100 | 86.11 | 7.56 |
| Carisoprodol | POS | 329.32 | 10.28 |
| Chlordiazepoxide | BZ 100 | 60.23 | 5.27 |
| Codeine | PM 100 | 84.28 | 6.31 |
| Cyclobenzaprine | POS | 152.42 | 4.18 |
| Desipramine | POS | 188.98 | 7.42 |
| Desmethyltramadol | POS | 330.79 | 9.18 |
| Desomorphine | POS | 316.45 | 8.60 |
| DesTapentadol | POS | 367.13 | 3.69 |
| Dextromethorphan | POS | 390.96 | 4.88 |
| Dextrorphan | POS | 302.44 | 6.63 |
| Diazepam | BZ 100 | 68.5 | 1.29 |
| Dihydrocodeine | POS | 264.34 | 9.32 |
| EDDP | PM 100 | 97.64 | 6.57 |
| Fentanyl | PM 100 | 11.36 | 7.49 |
| Flunitrazepam | BZ 100 | 80.49 | 7.86 |
| Gabapentin | POS | 1077.48 | 8.11 |
| Hydrocodone | PM 100 | 82.34 | 7.76 |
| Hydromorphone | PM 100 | 82.32 | 3.58 |
| Imipramine | POS | 180.64 | 5.29 |
| Lorazepam | BZ 100 | 100.59 | 4.64 |
| MDA | POS | 1392.91 | 12.30 |
| MDMA | POS | 1413.43 | 6.84 |
| Meperidine | POS | 275.67 | 5.75 |
| Meprobamate | POS | 348.37 | 6.07 |
| Methadone | C3 | 332.6 | 3.12 |
| Methamphetamine | POS | 1429.31 | 8.88 |
| Methylphenidate | POS | 127.81 | 5.11 |
| Midazolam | BZ 100 | 68.1 | 6.52 |
| Morphine | PM 100 | 96.13 | 4.18 |
| Naloxone | POS | 277.89 | 8.12 |
| Norbuprenorphine | PM 100 | 91.43 | 12.49 |
| Nordiazepam | BZ 100 | 83.01 | 4.28 |
| Norfentanyl | PM 100 | 13.25 | 4.89 |
| Normeperidine | POS | 340.25 | 8.69 |
| Norpropoxyphene | C3 | 203.78 | 9.01 |
| Nortriptyline | POS | 151.38 | 4.22 |
| Oxazepam | BZ 100 | 84.48 | 5.13 |
| Oxycodone | PM 100 | 84.11 | 9.17 |
| Oxymorphone | PM 100 | 83.94 | 3.81 |
| PCP | C3 | 27.26 | 6.14 |
| Pregabalin | POS | 295.75 | 9.69 |
| Propoxyphene | C3 | 329.14 | 9.77 |
| Ritalinic Acid | POS | 1360.81 | 4.48 |
| Sertraline | POS | 152.82 | 2.91 |
| Tapentadol | POS | 334.33 | 5.01 |
| Temazepam | BZ 100 | 76.93 | 3.91 |
| Tramadol | POS | 259.3 | 7.58 |
| Triazolam | BZ 100 | 64.97 | 4.14 |
| Zaleplon | POS | 355.86 | 12.71 |
| Zopiclone | POS | 223.53 | 10.72 |
| Zolpidem | POS | 263.9 | 4.90 |
| Carboxy Zolpidem | POS | 334.25 | 5.86 |
| Butalbital | C3 | 210.27 | 5.54 |
| THC | C3 | 10.81 | 6.80 |
| Phenobarbital | C3 | 213.69 | 7.88 |

TABLE 12
Inter-day study of recovery with the quality
controls and their yield (n = 10)

| | | Mean | CV |
|---|---|---|---|
| Component Name | QC Name | n = 10 | (%) |
| 6-MAM | C3 | 9.32 | 8.59 |
| OH-et-Flurazepam | BZ 100 | 79.38 | 4.68 |
| 4-Hydroxyalprazolam | BZ 100 | 83.49 | 4.98 |
| 7-Aminoclonazepam | BZ 100 | 112.24 | 6.68 |
| Amitriptyline | POS | 138.38 | 3.54 |
| Amphetamine | POS | 1558.05 | 8.54 |
| Benzoylecgonine | C3 | 196.79 | 2.91 |
| Buprenorphine | C3 | 0.27 | 42.23 |
| Carisoprodol | POS | 329.32 | 10.28 |
| Chlordiazepoxide | BZ 100 | 60.23 | 5.27 |
| Codeine | PM 100 | 84.28 | 6.31 |
| Cyclobenzaprine | POS | 152.42 | 4.18 |
| Desipramine | POS | 188.98 | 7.42 |
| Desmethyltramadol | POS | 330.79 | 9.18 |
| Desomorphine | POS | 316.45 | 8.60 |
| DesTapentadol | POS | 367.13 | 3.69 |
| Dextromethorphan | POS | 390.96 | 4.88 |
| Dextrorphan | POS | 302.44 | 6.63 |
| Diazepam | BZ 100 | 68.5 | 1.29 |
| Dihydrocodeine | POS | 264.34 | 9.32 |
| EDDP | PM 100 | 97.64 | 6.57 |
| Fentanyl | PM 100 | 11.36 | 7.49 |
| Flunitrazepam | BZ 100 | 80.49 | 7.86 |
| Gabapentin | POS | 1077.48 | 8.11 |
| Hydrocodone | PM 100 | 82.34 | 7.76 |
| Hydromorphone | PM 100 | 82.32 | 3.58 |
| Imipramine | POS | 180.64 | 5.29 |
| Lorazepam | BZ 100 | 100.59 | 4.64 |
| MDA | POS | 1338.53 | 9.05 |
| MDMA | POS | 1413.43 | 6.84 |
| Meperidine | POS | 275.67 | 5.75 |
| Meprobamate | POS | 348.37 | 6.07 |
| Methadone | C3 | 332.6 | 3.12 |
| Methamphetamine | POS | 1429.31 | 8.88 |
| Methylphenidate | POS | 127.81 | 5.11 |
| Midazolam | BZ 100 | 60 | 6.71 |
| Morphine | PM 100 | 96.13 | 4.18 |
| Naloxone | POS | 277.89 | 8.12 |
| Norbuprenorphine | C3 | 0.68 | 64.20 |
| Nordiazepam | BZ 100 | 83.01 | 4.28 |
| Norfentanyl | PM 100 | 13.25 | 4.89 |
| Normeperidine | POS | 351.23 | 6.38 |
| Norpropoxyphene | C3 | 203.78 | 9.01 |
| Nortriptyline | POS | 151.38 | 4.22 |
| Oxazepam | BZ 100 | 84.48 | 5.13 |
| Oxycodone | PM 100 | 84.11 | 9.17 |
| Oxymorphone | PM 100 | 83.94 | 3.81 |
| PCP | C3 | 27.26 | 6.14 |
| Pregabalin | POS | 295.75 | 9.69 |
| Propoxyphene | C3 | 329.14 | 9.77 |
| Ritalinic Acid | POS | 1360.81 | 4.48 |
| Sertraline | POS | 152.82 | 2.91 |
| Tapentadol | POS | 334.33 | 5.01 |
| Temazepam | BZ 100 | 76.93 | 3.91 |
| Tramadol | POS | 259.3 | 7.58 |
| Triazolam | BZ 100 | 64.97 | 4.14 |
| Zaleplon | POS | 355.86 | 12.71 |
| Zopiclone | POS | 223.53 | 10.72 |
| Zolpidem | POS | 263.9 | 4.90 |
| Carboxy Zolpidem | POS | 334.25 | 5.86 |
| Butalbital | C3 | 210.27 | 5.54 |
| THC | C3 | 10.81 | 6.80 |
| Phenobarbital | C3 | 213.69 | 7.88 |

An object of the presently disclosed subject matter having been stated hereinabove, and which is achieved in whole or in part by the presently disclosed subject matter, other objects and advantages will become evident to those of ordinary skill in the art after a study of the description of the presently disclosed subject matter, figures, and non-limiting examples.

We claim:

1. A method of detection and/or quantification of multiple drugs and/or metabolites from a sample of body fluid comprising the steps:
(a) mixing the sample with internal standard,
(b) hydrolyzing the drug metabolite in the sample by P-glucuronidase enzyme,
(c) centrifugation of the mixture of step (b) and diluting the clear supernatant liquid with deionized water, and
(d) analyzing said sample using liquid chromatography tandem mass spectrometer (LC-MS-MS) to determine the concentration of different drug metabolites;
wherein, the method is devoid of solid and/or liquid phase extraction and/or derivatization.

2. The method of claim 1, wherein said separation in liquid chromatography is performed by fast polarity switching.

3. The method of claim 1, wherein said biological sample is a bodily fluid selected from the group consisting of oral fluids (saliva), sweat, urine, blood, serum, plasma, spinal fluid, and combination thereof.

4. The method of claim 1, wherein the detection and/or quantification is employed simultaneously for the drugs belonging to different chemical and toxicological classes selected from the group, consisting of opiates/opioids, benzodiazepines, barbiturates, amphetamines, tricyclic antidepressants, illicit drugs, Z drugs and antiepileptics.

5. The method of claim 4, wherein opiates/opioids were selected from the group consisting of 6-monoacetylmorphine (6-MAM), codeine, dihydrocodeine, hydrocodone, hydromorphone, morphine, oxycodone, oxymorphone, buprenophrine, carisoprodol, desmethyl tapentadol, desmethyl tramadol, 2-ethylidene-1,5-dimethyl-3,3-diphenylpyrrolidine (EDDP), meperidine, meprobamate, methadone, norbuprenophrine, normeperidine, tapentadol, tramadol, fentanyl, norfentanyl, norpropoxyphene, propoxyphene, dextromethophan, dextrophan, desomorphine and nalaxone.

6. The method of claim 4, wherein benzodiazepines were selected from the group consisting of 7-aminoclonazepam, diazepam, flunitrazepam, 4-hydroxyalprazolam, nordiazepam, oxazepam, temazepam, chloradiazepoxide, OH-et-flunizepam, lorazepam, Triazolam and midazolam.

7. The method of claim 4, wherein barbiturates were selected from the group consisting of butalbital and phenobarbital.

8. The method of claim 4, wherein amphetamines were selected from the group consisting of amphetamine, 3,4-methylenedioxyamphetamine (MDA), 3,4-methylenedioxy-methamphetamine (MDMA), methamphetamine.

9. The method of claim 4, wherein tricyclic antidepressants were selected from the group consisting of desipramine, imipramine, nortriptyline, ritalinic acid, sertraline, cyclobenzaprine, amitriptyline and methyl phenidate.

10. The method of claim 4, wherein illicit drugs were selected from the group consisting of tetrahydrocannabinolic acid (THCA), benzoylecgonine and phencyclidine (PCP).

11. The method of claim 4, wherein Z-drugs were selected from the group consisting of zolpidem, zaleplon, zopiclone and zolpidem-COOH.

12. The method of claim 4, wherein antiepileptics were selected from the group consisting of pregabilan and gabapentin.

13. The method according to claim 1, wherein the sample of body fluid is analyzed for simultaneous detection and/or quantification of 6-monoacetylmorphine (6-MAM), codeine, dihydrocodeine, hydrocodone, hydromorphone, morphine, oxycodone, oxymorphone, buprenophrine, carisoprodol, desmethyl tapentadol, desmethyl tramadol, 2-ethylidene-1,5-dimethyl-3 ,3-diphenylpyrrolidine (EDDP), meperidine, meprobamate, methadone, norbuprenophrine, normeperidine, tapentadol, tramadol, fentanyl, norfentanyl, norpropoxyphene, propoxyphene, dextromethophan, dextrophan, desomorphine, nalaxone, 7-aminoclonazepam, diazepam, flunitrazepam, 4-hydroxyalprazolam, nordiazepam, oxazepam, temazepam, chloradiazepoxide, OH-et-flunizepam, lorazepam, triazolam, midazolam, butalbital, Phenobarbital, amphetamine, 3,4-methylenedioxyamphetamine (MDA), 3,4-methylenedioxy-methamphetamine (MDMA), methamphetamine, desipramine, imipramine, nortriptyline, ritalinic acid, sertraline, cyclobenzaprine, amitriptyline, methyl phenidate, tetrahydrocannabinolic acid (THCA), benzoylecgonine, phencyclidine (PCP), zolpidem, zaleplon, zopiclone, zolpidem-COOH, pregabilan and gabapentin.
